(19) United States
(12) Patent Application Publication
(10) Pub. No.: US 2021/0008117 A1
(43) Pub. Date: Jan. 14, 2021

(54) ADIPONECTIN-EXPRESSING REGULATORY T-CELL PRECURSORS, COMPOSITION AND METHODS THEREOF

(71) Applicant: THE UNIVERSITY OF HONG KONG, Hong Kong (CN)

(72) Inventors: Yu WANG, Hong Kong (CN); Aimin XU, Hong Kong (CN); Yiwei ZHANG, Hong Kong (CN); Handi CAO, Hong Kong (CN)

(21) Appl. No.: 16/864,774

(22) Filed: May 1, 2020

Related U.S. Application Data
(60) Provisional application No. 62/842,703, filed on May 3, 2019.

(51) Int. Cl. A61K 35/26; C12N 5/078; C07K 14/72; A01K 67/027

(52) U.S. Cl. CPC A61K 35/26; C12N 5/065; A01K 2227/105; A01K 67/027; A01K 2217/206; C07K 14/72

(57) ABSTRACT

Adiponectin-expressing thymocytes and compositions comprising the thymocytes are provided. Methods are provided for using the adiponectin-expressing thymocytes for metabolic regulation and treatment of hyperproliferative diseases.

### 1. FIELD

Adiponectin-expressing thymocytes and compositions comprising the thymocytes are provided. Methods are provided for using the adiponectin-expressing thymocytes for metabolic regulation and treatment of hyperproliferative diseases.

### SEQUENCE LISTING

The instant application contains a Sequence Listing which has been submitted electronically in ASCII format and is hereby incorporated by reference in its entirety. Said ASCII copy, created on Sep. 16, 2020, is named 10030_007699-US1 and is 5.76 KB in size.

### 2. BACKGROUND

Adiponectin is a ˜30 kDa glycoprotein that forms trimers, hexamers and high-molecular-weight (HMW) species (Wang et al., 2006b; Wang et al., 2005). Apart from adipose tissue, adiponectin has been identified as a factor produced from a subset of unstimulated non-B non-T lymphocytes to inhibit granulopoiesis (Crawford et al., 2010; Yokota et al., 2000). High levels of adiponectin are detected in the bone marrow, certain lymphoid cell lines and immune effector cells purified from healthy donors (Jasinski-Bergner et al., 2017). Adiponectin is expressed by components of the hemopoietic stem cell (HSC) niche to increase HSC proliferation, while retaining the cells in a functionally immature state (DiMascio et al., 2007).

Thymus is a major organ for the development and maturation of lymphocytes to produce multiple distinct subsets of T-cells (Takahama et al., 2017). The lymphoid progenitors migrate from the bone marrow into the circulation and then enter the thymus, in which they expand by forming the double negative (DN), double positive (DP) and single positive (SP) T-cells (Krueger et al., 2017). The microenvironment of thymus promotes the differentiation of lymphoid progenitors into T-lineage cells, the positive/negative selection of newly generated T-lymphocytes and the production of regulatory T-cells (Treg) for establishing self-tolerance (Klein et al., 2019).

Metabolic diseases including obesity and type 2 diabetes are rapidly rising in the modem society. Currently, there lacks effective treatments and regimens for patients with these diseases. Moreover, overweight and obesity will overtake smoking as the number one cause for cancer development. It is important to find a regimen to cure dietary obesity-induced metabolic diseases. It is also important to discover a method that effectively inhibit breast cancer development.

### 3. SUMMARY

Provided herein is a population of thymocytes that express adiponectin, an anti-diabetic, anti-inflammatory, anti-atherosclerotic and anti-cancer hormone. Adoptive transfer of these cells prevented the development of metabolic dysfunction in wild type mice fed with high fat diet, and inhibited breast cancer development in MMTV-PyVT transgenic mice. Adiponectin-expressing thymocytes regulate the development/selection of lymphocytes, the production of regulatory T-cells (Treg) in thymus, thus contributing to T-cell homeostasis in the body. The discovery provides a new approach of immunotherapy for metabolic and cancer diseases, by adoptive transfer of patient-derived (autologous) or donor-derived (allogenic) adiponectin-expressing thymocytes.

The present study demonstrates that adiponectin is expressed in a subpopulation of thymic progenitors that are able to develop into the mature thymic Treg (tTreg) cells. The adiponectin-expressing tTreg precursors are involved in the selection and development of T lymphocytes in the thymic nurse cell (TNC) complexes, in turn modulating the systemic T-cell homeostasis. Deficiency of adiponectin alters the maturation of tTreg and the selection of T lymphocytes in thymus, thus contributing to the development of diseases such as breast cancer and obesity-related metabolic complications.

Provided herein is a method of isolating adiponectin-expressing thymocytes from a subject comprising the steps of: (i) obtaining a portion of thymus from the subject; (ii) enriching thymic nurse cell (TNC) complexes from the thymus; (iii) preparing single-cell suspension from the TNC; and (iv) sorting adiponectin-expressing thymocytes from the single-cell suspension.

In certain embodiments, the adiponectin-expressing thymocytes located within a thymic nurse cell (TNC) complex expresses at least 4-folds adiponectin as compared to thymus tissue outside of the TNC.

In certain embodiments, the adiponectin-expressing thymocytes are characterized by high expression of CD117, CD4+, CD25+, and low expression of CD8−.

Provided herein is a method of treating autoimmune or inflammatory disease in a subject, said method comprising the steps of: (i) obtaining a portion of a thymus from a subject; (ii) enriching thymic nurse cell (TNC) complexes from the thymus; (iii) preparing single-cell suspension from the TNC; (iv) sorting adiponectin-expressing thymocytes from the single-cell suspension; and (v) administering an effective amount of adiponectin-expressing thymocytes to a subject in need thereof.

In certain embodiments, the thymus of step (i) was obtained from the same subject to be treated.

In certain embodiments, the thymus of step (i) was obtained from a different subject to be treated.

In certain embodiments, the method further comprising a step of culturing the adiponectin-expressing thymocytes after step (iv).

In certain embodiments, after the treatment, the subject has one or more of the following outcomes: (i) maintaining immune homeostasis; (ii) facilitating self-tolerance; (iii) inhibiting mammary tumor; (iv) inhibiting dietary obesity-induced gain of body fat mass; (v) improved glucose tolerance; (vi) reducing circulating lipid levels; (vii) enhanced energy expenditure; (viii) reducing atherosclerosis; (ix) hepatoprotection; (x) improved insulin sensitivity; (xi) enhanced oxygen consumption; (xii) enhanced CO2 production; (xiii) reduced circulating triglyceride and cholesterol levels; (xiv) reduced plasma concentrations of alanine transaminase (ALT); and (xv) reduced level of aspartate transaminase (AST).

In certain embodiments, the effective amount of adiponectin-expressing thymocytes is about 30,000 to about 500,000 cells.

Provided herein is a thymic nurse cell (TNC) complex comprising isolated adiponectin-expressing thymocytes from an Adn-Cre/ROSAmT/mG mouse wherein said adiponectin-expressing thymocytes express EGFP.

In certain embodiments, the thymocytes are characterized by high expression of CD117, CD4+, CD25+, and low expression of CD8−.

Provided herein is a method of producing adiponectin-rich thymocytes from an Adn-Cre/ROSAmT/mG mouse comprising the steps of: (i) obtaining a portion of a thymus from the mouse; (ii) enriching thymic nurse cell (TNC) complexes from the thymus; (iii) preparing single-cell suspension from the TNC; (iv) sorting adiponectin-expressing thymocytes EGFP⁺ cells from the single-cell suspension.

In certain embodiments, the thymocyte is derived from intrathymic precursor cells.

In certain embodiments, the thymocyte is a DN1 cell, DN1a, DN1b subpopulation, CD4SP cell or CD4+CD8+DP cell.

In certain embodiments, the thymocyte is characterized by high expression of CD117, CD4+CD25+CD8−.

Provided herein is a method of treating mammary tumor comprising a step of administering an effective amount of adiponectin-expressing thymocytes to a subject in need thereof.

Provided herein is a method of inhibiting dietary obesity-induced gain of body fat mass, improved glucose tolerance, reduced circulating lipid levels, and enhanced energy expenditure in a subject comprising a step of administering an effective amount of adiponectin-expressing thymocytes to the subject in need thereof.

Provided herein is a method of treating inflammation and atherosclerosis in a subject comprising a step of administering an effective amount of adiponectin-expressing thymocytes to the subject in need thereof.

In certain embodiments, the effective amount of adiponectin-expressing thymocytes is about 30,000 to about 500,000 cells.

In certain embodiments, the subject contained significantly increased amount of CD3⁺CD4⁺ T-cells and CD3⁺CD8⁺ T-cells.

Provided herein is a method of producing adiponectin-rich thymocytes from an Adn-Cre/ROSAmT/mG mouse comprising the steps of: (i) preparing single cell suspension from thymus of the Adn-Cre/ROSAmT/mG mouse; and (ii) sorting EGFP⁺ cells from the cell suspension of step (i).

Provided herein is the transgenic mouse Adn-Cre/ROSAmT/mG.

Provided herein is a method of treating or preventing an autoimmune, metabolic, hyperproliferative or inflammatory disease in a subject, said method comprising the steps of administering a therapeutically effective amount of thymocytes comprising a population of adiponectin-expressing regulatory T cell (Treg) to a subject in need thereof.

In certain embodiments, the adiponectin-expressing thymocytes are obtained by (i) obtaining a portion of a thymus from a subject; (ii) enriching thymic nurse cell (TNC) complexes from the thymus; (iii) preparing single-cell suspension from the TNC; and (iv) sorting adiponectin-expressing thymocytes from the single-cell suspension.

In certain embodiments, the adiponectin-expressing Treg precursors differentiate into regulatory T cells in the subject.

In certain embodiments, the metabolic disorder is selected from the group consisting of metabolic syndrome, type-1 diabetes mellitus, type-2 diabetes, obesity, diseases associated with an abnormal fat metabolism, gout disease (metabolic arthritis), hyperglycemia, hyperinsulinemia, insulin resistance, elevated blood levels of fatty acids or glycerol, syndrome X, and diabetic complications.

In certain embodiments, the autoimmune or inflammatory disorder is selected from the group consisting of wherein the autoimmune or inflammatory disease is selected from the group consisting of rheumatoid arthritis, multiple sclerosis, autoimmune hemolytic anemia, autoimmune oophoritis, autoimmune thyroiditis, autoimmune uveoretinitis, Crohn's disease, chronic immune thrombocytopenic purpura, colitis, contact sensitivity disease, type-1 diabetes mellitus, Graves disease, Guillain-Barre's syndrome, Hashimoto's disease, idiopathic myxedema, myasthenia gravis, psoriasis, pemphigus vulgaris, systemic lupus erythematosus, scleroderma, Reynaud's syndrome, Sjorgen's syndrome, autoimmune myocarditis, inflammatory bowel disease, Amyotrophic Lateral Sclerosis (ALS), Neuromyelitis Optica, Idiopathic Thrombocytopenic Purpura, Thrombotic Thrombocytopenic Purpura, Membranous Nephropathy, Bullous Phemphigoid, Phemphigus Vulgaris, Celiac disease, and ulcerative colitis.

In certain embodiments, the hyperproliferative disease is cancer, including metastases thereof.

In certain embodiments, the cancer is selected from the group consisting of ovarian cancer, prostate cancer, breast cancer, skin cancer, melanoma, colon cancer, lung cancer, pancreatic cancer, gastric cancer, bladder cancer, Ewing's sarcoma, lymphoma, leukemia, multiple myeloma, head and neck cancer, kidney cancer, bone cancer, liver cancer and thyroid cancer, including metastases thereof.

In certain embodiments, the thymocytes are obtained from the same subject to be treated.

In certain embodiments, the thymocytes are obtained from a different subject to be treated.

In certain embodiments, after the treatment, the subject has one or more of the following outcomes: (i) maintaining immune homeostatis; (ii) facilitating self-tolerance; (iii) inhibiting mammary tumor; (iv) inhibiting dietary obesity-induced gain of body fat mass; (v) improved glucose tolerance; (vi) reducing circulating lipid levels; (vii) enhanced energy expenditure; (viii) reducing atherosclerosis; (ix) hepatoprotection; (x) improved insulin sensitivity; (xi) enhanced oxygen consumption; (xii) enhanced CO2 production; (xiii) reduced circulating triglyceride and cholesterol levels; (xiv) reduced plasma concentrations of alanine transaminase (ALT); and (xv) reduced level of aspartate transaminase (AST).

In certain embodiments, the effective amount of adiponectin-expressing thymocytes is about 30,000 to about 500,000 cells.

In certain embodiments, the method further comprising a step of culturing the adiponectin-expressing thymocytes after step (iv).

### 4. DESCRIPTION OF THE FIGURES

The patent or application file contains at least one drawing executed in color. Copies of this patent or patent application publication with color drawings will be provided by the Office upon request and payment of the necessary fee.

FIGS. 1A-D. Adiponectin is expressed in thymus. A, Wild type [WT] or adiponectin knockout [AKO] mice were sacrificed at the age of seven weeks to collect epididymal adipose tissue [epid], liver and thymus. Adiponectin [Adn] protein expression was analyzed by denatured (left) or non-reducing (right) SDS-PAGE and detected by Western blotting using a polyclonal antibody recognizing murine adiponectin. Beta-actin (β-actin) was probed as loading controls. B, Immunofluorescence staining was performed to examine the expression and distribution of Adn protein [red] in the thymus of WT mice. The tissue sections were counterstained for CD31 [green] and DAPI [blue]. C and M indicate cortex and medulla, respectively. C, In situ hybridization was performed for detecting the mRNA transcripts of Adipoq in cell suspensions isolated from the thymus of WT mice. Positive brown signals were indicated by black arrows. D, Confocal fluorescence microscopy was applied to analyze the protein expression and distribution of Adn [green] in TNC complexes isolated from WT thymus. The sections were counterstained [red] for cytokeratin [CK] 5 and CK8.

FIGS. 2A-D. Flow cytometric analyses of adiponectin-expressing thymocytes. A, Tissue cryosections were prepared from thymus of ROSAmT/mG and Adn-Cre/ROSAmT/mG mice to visualize cells expressing the EGFP+ green fluorescence. B, Single cell suspensions were prepared from the thymus of Adn-Cre/ROSAmT/mG mice. Flow cytometry was performed to analyze EGFP+ cells and their surface expression of markers including CD45, CD326, CD4, CD8 and CD3. C, TNC complexes were isolated from the thymus of Adn-Cre/ROSAmT/mG mice and visualized by real-time live confocal microscopy [more details in Supplementary Online Video 1]. D, Flow cytometry was performed to analyze adiponectin-expressing EGFP+ cells in TNC complexes, and their surface expression of CD4, CD8, CD117 and CD25.

FIGS. 3A-D. Adiponectin-expressing thymocytes are tTreg precursors. A, EGFP+ cells collected from the thymus of Adn-Cre/ROSAmT/mG mice were injected [30000 cells/per mouse via tail vein] into WT or AKO recipient mice, which were subjected to a sub-lethal 5Gy γ-radiation prior to the injection. At twelve hours after adoptive transfer, cell suspensions were prepared from the thymus of the recipient mice for visualizing the EGFP+ cells (left) and immunofluorescence staining for adiponectin protein (right). B, At one day after cell injection, flow cytometry was performed to analyze the distribution of EGFP+ cells in the thymus of WT (left) and AKO (right) recipient mice, after staining with antibodies recognizing CD117, CD25, CD4 and CD8. C, At 15 days after injection, flow cytometry was performed to analyze the distribution of EGFP+ cells in the thymus of WT (left) and AKO (right) recipient mice, after staining with antibodies recognizing CD4, CD8, CD25 and Foxp3. D, The percentage contents and total numbers of EGFP+ cells in the thymus of WT and AKO recipient mice were calculated based on flow cytometric data collected from day one and 15 after adoptive transfer. Data are presented as mean±SEM. *, P<0.05 and **, P<0.001 vs corresponding WT controls (n=6).

FIGS. 4A-D. Treatment with adiponectin-expressing tTreg precursors alleviates HFD-induced metabolic abnormalities. Vehicle or adiponectin-expressing EGFP+ cells [30000 cells/mouse] isolated from Adn-Cre/ROSAmT/mG mice were injected via tail vein into four-weeks old WT mice, which were then subjected to HFD feeding for another 18-weeks. A, At the end of treatment, the gain of body weight and fat mass were calculated for comparison with the vehicle control group. B, After 12- and 14-weeks of HFD, intraperitoneal glucose (left) and insulin (right) tolerance tests were performed for comparison as described in the Methods. C, Indirect calorimetry was used to examine the VO2, VCO2, energy expenditure and RER as described in Methods for comparison. D, The fasting blood glucose and plasma insulin, triglyceride or cholesterol levels were measured after 16-18 weeks of HFD feeding for comparison. Data are presented as mean±SEM. *, P<0.05 vs corresponding vehicle control group (n=6).

FIGS. 5A-D. Hepatoprotection mediated by the adiponectin-expressing tTreg precursors. Vehicle or adiponectin-expressing EGFP+ cells [30000 cells/mouse] isolated from Adn-Cre/ROSAmT/mG mice were injected via tail vein into four-weeks old WT mice, which were then subjected to HFD feeding for another 18-weeks. At the end of treatment, blood, liver and epididymal adipose tissue were collected for subsequent analyses. A, Flow cytometry was performed to analyze the amount of CD3+CD4+ and CD3+CD8+ cells in the blood circulation for comparison. B, Flow cytometry was performed to compare the amount of Treg and Th17 cells in liver tissues for comparison. C, Hematoxylin and Eosin [H&E] or Oil Red O staining were performed on liver tissue sections to evaluate the accumulation and distribution of lipid droplets (left). The triglyceride and cholesterol contents in liver samples were examined by biochemical assays (middle). QPCR was performed for measuring the gene expression levels of PPARα and FGF21 in liver (right). D, QPCR was performed for measuring the gene expression levels of TNFα, MCP1 and Adipoq in epididymal adipose tissue. Data are presented as mean±SEM; *, P<0.05 vs corresponding vehicle controls (n=6).

FIGS. 6A-D. Treatment with adiponectin-expressing thymocytes inhibits breast cancer development in MMTV-PyVT mice. Vehicle or EGFP+ cells collected from the thymus of four to five-weeks old Adn-Cre/ROSAmt/mg mice were adoptively transferred into four-weeks old MMTV-PyVT mice via tail vein injection [30000 cells/per mouse]. Mammary tumor development was monitored once per week as described in Methods. At the age of 14-weeks, mice were sacrificed to collect blood, tumor and lung samples for analyses. A, The tumor growth, body weights, tumor and lung tissue weights were measured and calculated for comparison. B, Flow cytometry was performed to analyze the percentage composition of CD3+CD4+ and CD3+CD8+ T-cells in blood samples. C, Flow cytometry was performed to analyze the Treg population in mammary tumors based on the antibody staining of CD4, CD25, Foxp3 and Nrp1. The percentage composition of CD4+CD25highFoxp3+ and CD4+CD25lowFoxp3+ Treg cells were calculated for comparison. D, The mRNA expression levels of genes including TGFβ, MCP1, IL6 and VEGFα were measured in tumor samples by QPCR. Data were presented as mean±SEM. *, P<0.05 and **, P<0.01 vs corresponding vehicle controls (n=6).

FIG. 7A-D Adiponectin facilitates T-cell selection and development within the TNC complexes. A, WT and AKO mice were sacrificed at the age of six to seven-weeks. Thymus and TNC complexes were prepared to analyze the percentage of CD3+ cell population by flow cytometry. B, Four-weeks old WT mice were subjected to vehicle treatment or adoptive transfer with EGFP+ cells from thymus of Adn-Cre/ROSAmT/mG mice (30000 cells/per mouse via tail vein) as described in the Methods. At 21 days after the injection, thymus was collected and subjected to flow cytometry analyses for measuring the percentage contents of CD3+ or CD4+CD8− cells. C, WT and AKO mice were sacrificed as in panel A to prepare thymic cell suspensions. Flow cytometry was performed to analyze the total amount of CD326+β5t+ TNC complexes, those with high expression levels of propidium iodide [PI], or attached outside of the TNC complexes with positive eCD45 staining. D, WT and AKO mice were sacrificed as in panel A to prepare the thymic cell suspensions enriched with TNC complexes. Inverted microscope was used to visualize and manually count the number of thymocytes within each TNC complex (top). Immunofluorescence staining was performed to detect CD3 (middle) and β5t (bottom) signals in TNC complexes of WT and AKO mice. The slides were counterstained with DAPI.

FIGS. 8A-D. Adiponectin regulates the expression and distribution of CD100 within the TNC complexes. WT or AKO mice were sacrificed at the age of seven-weeks to collect the thymus for subsequent analyses. A, TNC complexes were prepared for immunofluorescence staining with antibodies against CD100, plexin B1 and CD72. Confocal microscopic analyses revealed different patterns of CD100 protein distribution in the preparations of TNC complexes from WT and AKO mice, respectively. B, Western blotting was performed to analyze the protein expression of CD100 in thymus and TNC lysates prepared from WT and AKO mice, using antibodies recognizing different regions [from amino acid 812-862 or 502-636] of this molecule. Beta-actin [β-actin] was detected as the loading controls. C, TNC complexes were prepared for co-immunoprecipitation using an antibody recognizing CD100 from amino acid 502-636. The presence of adiponectin and CD100 in the immune complexes was confirmed by Western blotting using specific antibodies (left). Immunofluorescence staining and confocal microscopic analyses were applied to analyze the interactions between CD100 and adiponectin within the TNC complexes (right). D, TNC complexes were prepared for co-immunoprecipitation using an antibody recognizing CD100 from amino acid 812-862. The presence of galectin-3 and CD100 in the immune complexes was confirmed by Western blotting using specific antibodies (left). Immunofluorescence staining and confocal microscopic analyses were applied to analyze the interactions between CD100 and galectin-3 within the TNC complexes (right).

FIGS. 9A-C. The mRNA transcripts of Adipoq were detected in thymus and TNC samples prepared from WT mice. Mice were sacrificed at the age of seven-weeks for analyzing the mRNA expression levels of Adipoq. A, RT-PCR was performed to detect the expression of full-length Adipoq transcript in epididymal fat [epid], liver and thymus tissues collected from WT or AKO mice. B, RT-PCR was performed to compare the expression of full-length Adipoq transcript in thymus and TNC samples of WT mice. C, QPCR was performed for measuring the relative mRNA expression levels of Adipoq in thymus and TNC samples of WT mice for comparison. Results are presented as fold changes. Data were presented as mean±SEM. *, P<0.001 vs thymus samples (n=6).

FIGS. 10A-C. Distribution of adiponectin-expression cells and adiponectin protein in TNC complexes. A, Adiponectin-expressing EGFP+ cells isolated from Adn-Cre/ROSAmT/mG mice were administered into four-weeks old WT mice [30000 cells/mouse via tail vein]. At 15-days after injection, TNC complexes were collected from thymus of the recepient mice for confocal microscopic analyses of the EGFP+ cells. Immunofluorescence counterstaining was performed detect neuropillin-1 [Nrp1] or galectin-3. B, Adiponectin-expressing EGFP+ cells isolated from Adn-Cre/ROSAmT/mG mice were administered into four-weeks old AKO mice [30000 cells/mouse via tail vein]. At 15-days after injection, TNC complexes were collected from thymus of the recepient mice for similar analyses as in Panel A. C, TNC complexes were isolated from six-weeks old WT mice to detect the protein distribution of adiponectin [Adn], Nrp1 and galectin-3 by immunofluorescent staining and confocal microscopic analyses.

FIGS. 11A-B. Detection of EGFP+ Treg cells in liver. WT mice were treated with adiponectin-expressing tTreg precursors as in FIGS. 4 and 5. A, Frozen tissue sections were prepared from liver samples to visualize the EGFP+ cells. B, Flow cytometry was performed to analyze the distribution of EGFP+ by staining with the markers including CD4, CD8, Foxp3 and Nrp1.

FIGS. 12A-B Altered T-cell repertoire in blood of mice lacking the expression of adiponectin. A, PyVT-WT or PyVT-AKO mice were sacrificed at the age of seven-weeks. Flow cytometry was performed to analyze the populations of CD3+CD4+, CD3+CD8+ and CD4+CD8+ cells in the blood circulation for comparison. B, PyVT-AKO mice were sacrificed at the age of seven-weeks. The CD4+CD8+ DP cells were collected from the blood samples and implanted (3000 cells/mouse) into NOD/SCID immune deficient mice together with 2×105 human breast cancer MDA-MB-231 cells. Compared with those only implanted with the same number of MDA-MB-231 cells [control], co-implantation with DP cells significantly enhanced the tumor development. Data were shown as means±SEM. *, P<0.05 vs corresponding controls (n=6-7).

FIG. 13. Altered epithelial microenvironment in thymus of mice lacking the expression of adiponectin. WT or AKO mice were sacrificed at the age of seven-weeks to collect the thymus. Immunofluorescence staining was performed to examine the expression and distribution of epithelial makers, including p63, galectin-3, β5t and cytokeratin 5 [CK5].

### 5. DETAILED DESCRIPTION

Adiponectin is an insulin sensitizer and anti-inflammatory molecule, possessing therapeutic potentials in obesity-related cardiovascular, metabolic and cancer diseases. It was originally discovered in adipocyte and considered as an abundant adipokine. Results of the present study demonstrate that adiponectin is expressed in a population of regulatory T-cells (Treg) in thymus. After tail vein injection, adiponectin-expressing Treg precursors rapidly reside within the thymic nurse cell (TNC) complexes and differentiate into thymic Treg cells (tTreg). The development of adiponectin-expressing tTreg precursors is significantly attenuated in thymus of mice without adiponectin (AKO). Adoptive transfer of adiponectin-expressing tTreg precursors effectively prevented obesity, glucose and insulin intolerance, as well as NAFLD in wild type mice (WT) fed with high fat diet, and significantly inhibited breast cancer development in MMTV-PyVT transgenic mice. Treatment with adiponectin-expressing tTreg precursors facilitated the selection and maturation of T lymphocytes in thymus and altered the T-cell repertoire in the peripheral organs. Within the TNC complexes, the locally produced adiponectin bound to CD100 and regulated the expression as well as the distribution of this transmembrane lymphocyte semaphorin. In summary, adiponectin-expressing tTreg precursors are critically involved in the selection and development of T-cells within the TNC complexes and represent a promising candidate for adoptive cell immunotherapy against obesity-related metabolic and cancer diseases.

Provided herein, adiponectin was expressed in a subset of Treg precursors in thymus, which regulated the T-cell development and maturation within the specialized environment of the thymic nurse cell complexes (TNCs). Adiponectin deficiency caused defective selections of lymphocytes and an excessive release of immature CD4+CD8+ DP cells from thymus into the circulation. The latter promoted tumor development in MMTV-PyVT transgenic mice. Treatment with adiponectin-expressing thymocytes not only inhibited the development of mammary tumors in MMTV-PyVT animals, but also prevented dietary obesity-induced metabolic disorders in wild type mice.

### 5.1 Thymocytes

Thymocytes” are developing T cells that are located in the thymus. Any particular thymocyte may be at one of several stages of development. Stages of thymocyte development may be distinguished by the expression of the surface protein markers called clusters of differentiation (CD). Expression of CD4 and CD8 markers are particularly useful for distinguishing various stages of thymocyte development. The least mature thymocytes do not express either CD4 or CD8 and are called “double negative” (or DN) cells. DN cells are found predominantly in the subcapsular and outer cortical regions of the thymus. DN cells may be further characterized as DN1, DN2, DN3 or DN4 thymocytes on the basis of other phenotypic markers, including for example CD25 and CD44. In particular, DN1 cells may be identified as CD25⁻/CD44⁺, DN2 cells may be identified as CD44⁺/CD25⁺, DN3 cells may be identified as CD25⁺/CD44⁻, and DN4 cells may be identified as CD44⁻/CD25⁻.

As thymocyte development progresses, thymocytes migrate into the cortex and begin to express both CD4 and CD8. CD4⁺/CD8⁺ thymocytes may be called “double positive” (or DP) cells. DP cells become responsive to antigens and are subject to positive and negative selection. Cells that successfully undergo selection then mature into CD4⁺/CD8⁻ or CD4⁻/CD8⁺ cells, which are also called “single positive” (or SP) cells. Single positive cells enter the thymic medulla and then leave the thymus, as mature T cells, to populate the peripheral lymphoid tissues.

In humans, circulating CD34+ hematopoietic stem cells (HSC) reside in bone marrow. They produce precursors of T lymphocytes, which seed the thymus (thus becoming thymocytes) and differentiate under influence of the Notch and its ligands. Early, double negative thymocytes express (and can be identified by) CD2, CD5 and CD7. Still during the double negative stage, CD34 expression stops and CD1 is expressed. Expression of both CD4 and CD8 makes them double positive and matures into either CD4+ or CD8+ cells.

Thymocytes are ultimately derived from bone marrow hematopoietic progenitor cells which reach the thymus through the circulation. The number of progenitors that enter the thymus each day is thought to be extremely small. Therefore, which progenitors colonize the thymus is unknown. Currently Early Lymphoid Progenitors (ELP) are proposed to settle the thymus and are likely the precursors of at least some thymocytes. ELPs are Lineage-CD44+CD25-CD117+ and thus closely resemble ETPs, the earliest progenitors in the thymus. Precursors enter the thymus at the cortico-medullary junction. Molecules known to be important for thymus entry include P-selectin (CD62P), and the chemokine receptors CCR7 and CCR9. Following thymus entry, progenitors proliferate to generate the ETP population. This step is followed by the generation of DN2 thymocytes which migrate from the cortico-medullary junction toward the thymus capsule. DN3 thymocytes are generated at the subcapsular zone. In addition to proliferation, differentiation and T lineage commitment occurs within the DN thymocyte population. Commitment, or loss of alternative lineage potentials (such as myeloid, B, and NK lineage potentials), is dependent on Notch signaling, and is complete by the DN3 stage. Following T lineage commitment, DN3 thymocytes undergo (3-selection.

### 5.1.1 Modulating Thymocyte Number

In certain embodiment, the number of thymocytes may be modulated in a subject prior to being isolated in the subject. To change the number of thymocytes present in the thymus of a subject as compared to a control time point in the same subject or as compared to a second subject that serves as a control. Thymocyte number in either control circumstance being referred to as “the control number of thymocytes.” Modulating thymocyte number encompasses increasing or decreasing thymocyte numbers from the control number of thymocytes. Where expressly indicated, modulating thymocyte number may refer to changing the number of a particular subset of thymocytes, for example, as in “modulating the number of DN thymocytes.

Increasing thymocyte number means resulting in more thymocytes as compared to the control number of thymocytes; for example, thymocyte numbers may be at least 10%, at least 25%, at least 50%, at least 100% or at least 250% higher than control, or in some examples even at least 10% higher than control.

A single-cell suspension is prepared from the thymus to isolate thymic CD4⁺ cells after depletion of CD8⁺ cells. T_{reg} are isolated by magnetic cell separation or flow cytometric sorting, expanded using anti-CD3/CD28 or artificial antigen-presenting cells, interleukin (IL)-2 and rapamycin. After expansion, T_{reg} are cultured, modified or cryopreserved accordingly.

### 5.2 Thymic Nurse Cell

Thymic nurse cells (TNC) are large epithelial cells found in the cortex of the thymus and also in cortico-medullary junction. They have their own nucleus and are known to internalize thymocytes through extensions of plasma membrane. The cell surfaces of TNCs and their cytoplasmic vacuoles express MHC Class I and MHC Class II antigens. The interaction of these antigens with the developing thymocytes determines whether the thymocytes undergo positive or negative selection.

Thymic nurse cells are a sub-population of cortical thymic epithelial cells (cTECs). pH91, which is a TNC-specific monoclonal antibody, that can be used to identify TNCs. Thymic nurse cells express both MHC Class I and II antigens, and are found in the cortico-medullary junction in addition to the cortex of the thymus. The thymic nurse cells in the cortico-medullary junction express cytokeratin 5 (K5) and cytokeratin 8 (K8), whereas the ones in the cortex express only cytokeratin 8. The extensions of plasma membrane from thymic nurse cells form a cage-like structure, which trap triple positive T cells, αβTCR^{low}CD4⁺CD8⁺ within the spaces formed by the interlocking of the membrane. Some of these T cells retain their mobility and undergo maturation to the developmental stage of αβTCR^{high}CD69⁺; they are then released from the TNC complex. The enclosed thymocytes have been found to remain intact and retain both metabolic and mitotic activities despite lacking any contact with the extracellular environment. Although initially thought to be involved only in positive selection, thymic nurse cells have now been discovered to facilitate negative selection of thymocytes as well. Negative selection refers to the degradation of thymocytes, and has been found to occur through the help of lysosomes. Lysosomes are present near the nucleus in the cytoplasm of TNCs. If the internalized thymocytes are selected for negative selection, vacuoles containing the thymocytes move closer to the area with lysosomes and eventually fuse with the lysosomes. This leads to the degradation of the T cells within the vacuoles. Macrophages have also been found actively moving in and out of the vacuoles inside the TNCs during the times of high apoptotic activity suggesting their involvement in the elimination of negatively selected T lymphocytes.

The thymic cortical cells take up early thymocytes migrating from the bone marrow to the thymus and form the thymocyte-TNC complexes. The formation of finger-like projections has been found to facilitate this uptake; which also requires the participation of membrane and cytoskeleton proteins of TECs and thymocytes.

### 5.2.1 Thymocytes within TNC

Incubation of TNC at 37° C. in tissue culture releases thymocytes (TNC-T) present within it. Incubation of TNC at 4° C. or room temperature inhibits release of TNC-T. Incubation of TNC at 37° C. in presence of 0.1% sodium azide prevents the release of TNC-T from within even though the TNC-T are viable. This suggests that metabolic activity of epithelial thymocyte complex is essential for the release of TNC-T. TNC-T are functionally mature than those external to TNC (ET). Based on the observation that TNC harbor functionally mature population of TNC-T and the electronmicroscopic studies suggesting that TNCs are localized in close proximity of blood capillaries in both cortex and cortico-medullary region of thymus,

### 5.3 Methods of Treatment

Provided herein are methods of treating insulin resistance-related diseases such as of metabolic syndrome, diabetes, hyperlipidemia, fatty liver disease, hypertension, obesity, and arteriosclerosis. In some embodiments, the fatty liver disease can be alcoholic fatty liver disease and non-alcoholic fatty liver disease. The method can effectively treat and improve symptoms or conditions of the subject affected with the disease by administering thymocytes that express adiponectin without causing adverse side-effects. Furthermore, the method can increase the adiponectin in the subject by 10-20%, 20-30%, 30-40%, 40-50%, 50-60%, 60-70%, 70-80%, 80%-90%, 90-100% more than before the administration. These methods of the present teachings can be expected to treat or improve the symptoms or conditions of the subject affected by the diseases or disorder.

Obesity and diabetes are closely related and their development is physiologically and metabolically regulated involving different tissues (intestine, liver, heart, muscle, adipose and pancreas) and multiple inter-related mechanisms. Conditions including (1) enhancement of intestinal glucose absorption, (2) reduction of glucose and fat uptake by muscle and heart, acceleration of lipid synthesis and storage in adipose tissue, (3) an increase in the number of adipocytes (fat cells), and (4) slowing thermogenesis all promote both diabetes and obesity. In contrast, conditions that (1) inhibit glucose absorption, (2) promote glucose and fat uptake by muscle and heart tissues, (3) suppress fat, cholesterol and glucose synthesis by the liver, (4) reduce the number of adipocytes, and (5) promote thermogenesis are beneficial in treating or preventing diabetes and obesity. [See e.g., Wang S, et al. (2014). “Novel insights of dietary polyphenols and obesity”. J. Nutr. Biochem., 25(1): 1-18; Centers for Disease Control and Prevention. “The Health Effects of Overweight and Obesity”. https://www.cdc.gov/healthyweight/effects/; Aguirre L, et al (2011), “Beneficial Effects of Quercetin on Obesity and Diabetes”. The Open Nutraceuticals Journal, 4: 189-198; Zhang D, et al. (2013), “Curcumin and Diabetes: A Systematic Review”. Evidence Based Complementary and Alternative Medicine; http://dx.doi.org/10.1155/2013/636053; Babu P V A, et al. (2013), “Recent Advances in Understanding the Anti-Diabetic Actions of Dietary Flavonoids”. J. Nutr. Biochem. Doi: 10.1016/jnutbio.2013.06.003.]

The present methods may treat metabolic disease including a wide range of diseases and disorders of the endocrine system including, for example, insulin resistance, diabetes, obesity, impaired glucose tolerance, high blood cholesterol, hyperglycemia, dyslipidemia and hyperlipidemia.

The present methods may treat or ameliorate an inflammation such as systemic inflammatory conditions and conditions associated locally with migration and attraction of monocytes, leukocytes and/or neutrophils. Examples of inflammation include, but are not limited to, Inflammation resulting from infection with pathogenic organisms (including gram-positive bacteria, gram-negative bacteria, viruses, fungi, and parasites such as protozoa and helminths), transplant rejection (including rejection of solid organs such as kidney, liver, heart, lung or cornea, as well as rejection of bone marrow transplants including graft-versus-host disease (GVHD)), or from localized chronic or acute autoimmune or allergic reactions. Autoimmune diseases include acute glomerulonephritis; rheumatoid or reactive arthritis; chronic glomerulonephritis inflammatory bowel diseases such as Crohn's disease, ulcerative colitis and necrotizing enterocolitis; granulocyte transfusion associated syndromes; inflammatory dermatoses such as contact dermatitis, atopic dermatitis, psoriasis; systemic lupus erythematosus (SLE), autoimmune thyroiditis, multiple sclerosis, and some forms of diabetes, or any other autoimmune state where attack by the subject's own immune system results in pathologic tissue destruction. Allergic reactions include allergic asthma, chronic bronchitis, acute and delayed hypersensitivity. Systemic inflammatory disease states include inflammation associated with trauma, burns, reperfusion following ischemic events (e.g. thrombotic events in heart, brain, intestines or peripheral vasculature, including myocardial infarction and stroke), sepsis, ARDS or multiple organ dysfunction syndrome. Inflammatory cell recruitment also occurs in atherosclerotic plaques. Inflammation includes, but is not limited to, Non-Hodgkin's lymphoma, Wegener's granulomatosis, Hashimoto's thyroiditis, hepatocellular carcinoma, thymus atrophy, chronic pancreatitis, rheumatoid arthritis, reactive lymphoid hyperplasia, osteoarthritis, ulcerative colitis, papillary carcinoma, Crohn's disease, ulcerative colitis, acute cholecystitis, chronic cholecystitis, cirrhosis, chronic sialadenitis, peritonitis, acute pancreatitis, chronic pancreatitis, chronic Gastritis, adenomyosis, endometriosis, acute cervicitis, chronic cervicitis, lymphoid hyperplasia, multiple sclerosis, hypertrophy secondary to idiopathic thrombocytopenic purpura, primary IgA nephropathy, systemic lupus erythematosus, psoriasis, pulmonary emphysema, chronic pyelonephritis, and chronic cystitis.

A treatment may prevent and/or slow the development of a targeted pathologic condition or disorder. The treatment may be curative, therapeutic or disease-modifying, including therapeutic measures that cure, slow down, lessen symptoms of, and/or halt progression of a diagnosed pathologic condition or disorder. A treatment may be for patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and to alleviate are also intended to include the potentiation or otherwise enhancement of one or more primary prophylactic or therapeutic measures. In certain embodiments, the disclosed method is useful for preventing cancer or certain disorder in subjects who are predisposed to cancer or disorder.

The subject including humans or non-human animals. the present teachings can be administered to a human or another animal. Non-limiting examples of other animals include mammals such as cows, pigs, horses, sheep, goats, donkeys, monkeys, dogs, cats, rabbits, mice, rats or guinea pigs. The present teachings can be administered to or ingested by a human, livestock, experimental animal or pet.

### 5.4 Cancer Treatment

Tumor growth and disease progression in a subject in conjunction with ACT may be monitored during and after treatment of cancer via the subject methods of the present invention. Clinical efficacy can be measured by any method known in the art. In some embodiments, clinical efficacy of the subject treatment method is determined by measuring the clinical benefit rate (CBR). In some embodiments, the clinical benefit rate is measured by determining the sum of the percentage of patients who are in complete remission (CR), the number of patients who are in partial remission (PR) and the number of patients having stable disease (SD) at a time point at least 6 months out from the end of therapy. In some embodiments, CBR for the subject treatment method is at least about 50%. In some embodiments, CBR for the subject treatment method is at least about 55%, 60%, 65%, 70%, 75%, 80%, 85%, 90%, 95% or more.

Cancers and related disorders that can be prevented, treated, or managed in accordance with the methods described herein include, but are not limited to, the following: Examples of tumors include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Additional cancers which can be treated by the disclosed composition according to the invention include but not limited to, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In one embodiment, the cancer is benign, e.g., polyps and benign lesions. In other embodiments, the cancer is metastatic, ACT can be used in the treatment of pre-malignant as well as malignant conditions. Pre-malignant conditions include hyperplasia, metaplasia, and dysplasia. Treatment of malignant conditions includes the treatment of primary as well as metastatic tumors. In a specific embodiment, the cancer is melanoma, colon cancer, renal cell carcinoma, or lung cancer (e.g., non-small cell lung cancer). In certain embodiments, the cancer is metastatic melanoma, metastatic colon cancer, metastatic renal cell carcinoma, or metastatic lung cancer (e.g., metastatic non-small cell lung cancer).

### 5.5 Combination Therapy

Cells, e.g., T cells, for adoptive cell transfer may also be administered with other therapeutic agents, e.g., a chemotherapeutic agent or a biological agent. Examples of chemotherapeutic agents which can be used in the compositions and methods of the invention include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents that can be used in the compositions and methods of the invention include monoclonal antibodies (e.g., rituximab, cetuximab, panetumumab, tositumomab, trastuzumab, alemtuzumab, gemtuzumab ozogamicin, and bevacizumab), enzymes (e.g., L-asparaginase), growth factors (e.g., colony stimulating factors and erythropoietin), cancer vaccines, gene therapy vectors, or any combination thereof.

Combination therapy performed with ACT includes concurrent and successive administration of an additional therapy with ACT. As used herein, the additional therapy and ACT are said to be administered concurrently if they are administered to the patient on the same day, for example, simultaneously, or 1, 2, 3, 4, 5, 6, 7, or 8 hours apart, whereas the additional therapy and ACT are said to be administered successively if they are administered to the patient on the different days, for example, administered at a 1-day, 2-day or 3-day intervals. In the methods described herein, administration of the additional therapy can precede or follow ACT. In some embodiments, administration of the additional therapy occurs before administration of ACT cells, e.g., at least 1 day before administration of ACT cells, or at least 2, at least 3, at least 4, at least 5, at least 6, or at least 7 days before administration of ACT cells. In some embodiments, administration of the additional therapy occurs after administration of ACT cells, e.g., at least 1 day after administration of ACT cells, or at least 2, at least 3, at least 4, at least 5, at least 6, or at least 7 days after administration of ACT cells.

### 5.6 Methods of Administration

In the methods of the disclosure, “administration” is not limited to any particular delivery system and may include, without limitation, parenteral (including subcutaneous, intravenous, intramedullary, intraarticular, intramuscular, or intraperitoneal injection), rectal, topical, transdermal, or oral (for example, in capsules, suspensions, or tablets). Administration to an individual may occur in a single dose or in repeat administrations, and in any of a variety of physiologically acceptable salt forms, and/or with an acceptable pharmaceutical carrier and/or additive as part of a pharmaceutical composition. Physiologically acceptable salt forms and standard pharmaceutical formulation techniques and excipients are well known to persons skilled in the art (see, e.g., Physicians' Desk Reference (PDR®) 2005, 59.sup.th ed., Medical Economics Company, 2004; and Remington: The Science and Practice of Pharmacy, eds. Gennado et al. 21th ed., Lippincott, Williams & Wilkins, 2005).

Intravascular cell delivery routes require that the cells reach their target site, remain there and are able to migrate through the endothelial layers. Stem cells have the capacity to migrate and need in addition to initiate a sequential process of recognition and firm adhesion. In certain embodiments, the cells are engrafted to the target site. In general, the administered cells are autologous/syngeneic, allogeneic or xenogenic.

### 5.7 Adoptive Cell Therapy

Adoptive cell therapy (ACT) is a treatment method where cells are removed from a donor, cultured and/or manipulated in vitro, and administered to a patient for the treatment of a disease. In some embodiments, cells administered in adoptive cell transfer are thymocytes. In some embodiments, the cells used for ACT are derived from the subject receiving ACT.

Whether a cell or cell population is positive for a particular cell surface marker can be determined by flow cytometry using staining with a specific antibody for the surface marker and an isotype matched control antibody. A cell population negative for a marker refers to the absence of significant staining of the cell population with the specific antibody above the isotype control, positive refers to uniform staining of the cell population above the isotype control. In some embodiments, a decrease in expression of one or markers refers to loss of 1 log 10 in the mean fluorescence intensity and/or decrease of percentage of cells that exhibit the marker of at least 20% of the cells, 25% of the cells, 30% of the cells, 35% of the cells, 40% of the cells, 45% of the cells, 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 20 and 100% when compared to a reference cell population. In some embodiments, a cell population positive for a marker refers to a percentage of cells that exhibit the marker of at least 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 50 and 100% when compared to a reference cell population.

A dose of the cells used in adoptive cell transfer can be administered to a mammal, e.g., a human, at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of thymocytes may be administered in a single daily dose, or the total daily dosage may be administered in two, three, four, or more divided doses administered daily, as needed.

With respect to an upper limit on the number of T cells that can be administered or the number of times that the T cells of the invention can be administered, one of ordinary skill in the art will understand that excessive quantities of administered T lymphocytes can lead to undesirable side effects and unnecessarily increase costs.

Cells for ACT, e.g., thymocytes, administered in accordance with the disclosure may be modified to express other polypeptides, such as chimerica antigen receptors and the like. In some embodiments, a preparation comprising cells for ACT does not substantially contain any other living cells. Adoptive cell therapy was applied to cancer therapy in human with anticancer immune cells from cancer-bearing host ^{(Rosenberg and Restifo, 2015)}.

### 5.8 Pharmaceutical Composition

Examples of nontoxic pharmaceutical carriers used in formulation include sugars such as glucose, lactose, sucrose, fructose or reduced maltose, carbohydrates such as starch, hydroxyethyl starch, dextrin, .beta.-cyclodextrin, crystalline cellulose or hydroxypropyl cellulose, sugar-alcohols such as mannitol, erythritol, sorbitol or xylitol, esters such as fatty acid glycerides or polyoxyethylene sorbitan fatty acid esters, polyethylene glycol, ethylene glycol, amino acids, albumin, casein, silicon dioxide, water and physiological saline. In addition, commonly used additives, such as stabilizers, lubricants, humectants, emulsifiers, suspending agents, binders, disintegration agents, solvents, solubilizing agents, buffers, isotonic agents, antiseptics, correctives or colorants. The carriers can be suitably added as necessary for the formulation. The dosage amount of the present teachings can be suitably selected or determined according to species, age (such as monthly age), body weight, symptoms or severity of disease of a human or animal to be administered the enhancer as well as the administration schedule or type of the formulation.

### 6. MATERIALS AND METHODS

### 6.1 Animal Experiment

### 6.1.1. Mouse Models

All mouse models were housed in a room under the controlled temperature (23±1° C.) and 12-h light-dark cycles, with free access to water and standard mouse chow (4.07 kcal/g; LabDiet 5053; LabDiet, Purina Mills, Richmond, Va., U.S.A.). All experimental procedures were approved by the Committee on the Use of Live Animals in Teaching and Research, the University of Hong Kong and carried out in compliance with the Guide for the Care and Use of Laboratory Animals published by the National Institutes of Health (8^{th} Edition, 2011; https://www.ncbi.nlm.nih.gov/books/NBK54050/).

Mice with (WT) or without (AKO) wild type Adipoq alleles were maintained on both C57BL/6J and FVB/N background. For metabolic evaluations, WT and AKO of C57BL/6J background were fed with high-fat diet (19.33 kcal/g from 49.85% fat, 20% protein, and 30.15% carbohydrate; D12451; Research Diet, New Brunswick, N.J., U.S.A.) to induce dietary obesity ^{(Lin et al., 2013, Zhou et al., 2010)}. For studies related to mammary tumor development, FVB/N-Tg (MMTV-PyVT)634 Mul/J [002374 from Jackson Laboratory (Bar Harbor, Me., U.S.A.)] were cross-bred with AKO of FVB/N background to produce mice with (PyVT-WT) or without (PyVT-AKO) the Adipoq alleles ^{(Lam et al., 2009, Liu et al., 2013)}. ROSA^{mT/mG} (007676; Jackson Laboratory) mice were crossbred with Tg(Adipoq-cre)1Evdr/J (010803; Jackson Laboratory) to obtain the transgenic two-color reporter mouse model, referred to as Adn-Cre/ROSA^{mT/Mg}. The Adn-Cre/ROSA^{mT/mG} mice were crossbred with AKO to produce Adn-Cre/ROSA^{mT/mG}-AKO mice lacking the expression of adiponectin. The genotyping primers for the above mouse models are listed in Table 1.

TABLE 1
List of primers used for genotyping PCR.

| Mouse | Primer sequences | Product |
|---|---|---|
| Adipoq | (F) 5′-CCAGAGAACAACGAACAAGGA-3′ | 473 bp |
| | (R) 5′-CGAATGGGTACATTGGGAAC-3′ | |
| Neo | (F) 5′-TGAATGAACTGCAGGACGAG-3′ | 171 bp |
| | (R) 5′-ATACTTTCTCGGCAGGAGCA-3′ | |
| MMTV- | (F) 5′-GGAAGCAAGTACTTCACAAGGG-3′ | 556 bp |
| PyVT | (R) 5′-GGAAAGTCACTAGGAGCAGGG-3′ | |
| Adipoq- | (F) 5′-GGATGTGCCATGTGAGTCTG-3′ | 200 bp |
| cre | (R) 5′-ACGGACAGAAGCATTTTCCA-3′ | |
| Gt(Rosa) | Common 5′-CTTTAAGCCTGCCCAGAAG | 212 bp |
| 26sor | A-3′ | (wild |
| (Neo) | Mutant (F) 5′-TAGAGCTTGCGGAACCCT | type) |
| | TC-3′ | 128 bp |
| | Wild type (F) 5′-AGGGAGCTGCAGTGG | (mutant) |
| | AGTAG-3′ | |

### 6.1.2 Intravenous Transfer

Cells expressing EGFP fluorescence (EGFP⁺) were freshly sorted from the thymus of Adn-Cre/ROSA^{mT/mG} mice. Around 3×10⁴ EGFP cells were injected via tail vein into four to five-weeks old WT and AKO, or PyVT-WT and PyVT-AKO mice. The WT and AKO mice were subsequently fed with high fat diet for another 18-weeks, during which their body weight, fat mass composition and insulin sensitivities were monitored on a weekly basis ^{(Xu et al., 2015)}. Tumor development in PyVT-WT and PyVT-AKO mice were monitored on a weekly basis as described ^{(Lam et al., 2009; Wang et al., 2006a)}. For evaluating the EGFP⁺ cell identities and development, the recipient mice were given 500 rad (5Gy) irradiation for four-six hours before the adoptive transfer.

### 6.1.3 Orthotopic Inoculation of Human Breast Cancer Cells

Nonobese diabetic/severe combined immunodeficient NOD.CB17-Prkdc^{scid}/J (NOD/SCID; 001303) mice were obtained from the Jackson Laboratory. Mice were anesthetized with a mixture of ketamine (90 mg/kg ip), xylazine (20 mg/ml ip) and acepromazine (1.8 mg/kg ip). Human breast cancer MDA-MB-231 cells (2×10⁵), together without or with the CD4⁺CD8⁺ cells (3×10³), were inoculated into the right thoracic mammary fat pads. The development of mammary tumors was monitored twice per week with a vernier caliper and calculated using the formula [sagittal dimension (mm)×cross dimension (mm)²]/2.

### 6.2 Flow Cytometry and Fluorescence-Activated Cell Sorting (FACS)

### 6.2.1 Instrument

Multicolor flow cytometry and cell sorting were performed with BD LSR Fortessa Analyzer (BD Bioscience, San Jose, Calif., U.S.A.) and BD FACSAria™ SORP Cell Sorter (BD Bioscience), respectively. The cytometer performance was checked prior to each experiment. The fluorescent lights activated by a solid-state laser of 405 nm (FVD eFluor 506, VioBlue), 488 nm (FITC, PE, PerCP, PE-Vio770), and 640 nm (APC, APC-Vio770) were collected by photomultiplier tubes, voltage of which was adjusted using the cell sample and fixed for all experiments. The fluorescence compensation was performed using a single-antibody-labeled COMPtrol Goat anti-Mouse Ig (H&L) Particle Kit (Spherotech, Lake Forest, Ill., U.S.A.) and then with the single-antibody-labeled cell samples. For each sample, at least 10,000 events were acquired for analyses. Data analyses were performed using Diva 6.1 and CellQuest (BD Biosciences) and FlowJo 10.0 software (TreeStar Inc., Ashland, Oreg., USA). Dead events were excluded by FSC-A/SSC-A gating and adhesion events excluded by FSC-A/FSC-H gating.

Adhesion events were excluded. The initial centralised gate was set on the brightest part of a pseudocolor plot. The initial extended gate was set to include the peripheral of the brightest part. Based on these two methods, centralised and extended scales were attempted. Thus, FSC/SSC (central), FSC/SSC (extended), FSC/Vt (central), and FSC/Vt (extended) gates were applied to quantify live cell subsets as percentages of the parental population.

### 6.3 Preparation of Thymic Cell Suspension

Single cell suspension was prepared from thymus as described ^{(Xing and Hogquist, 2014)}. In brief, freshly collected tissues were cut, minced and transferred to Dulbecco's Modified Eagle Medium (DMEM) containing 2 mg/ml collagenase Type I (Gibico™, Waltham, Mass., U.S.A.) and 40 m/ml DNase I (Sigma-Aldrich, St. Louis, Mo., U.S.A.). After incubation at 37° C. with shaking for 30 minutes, cells were strained through a 100 μm filter mesh and centrifuged at 400×g. The pellets were re-suspended in phosphate-buffered saline (PBS) and then labeled with specific antibodies for subsequent flow cytometric analyses. Where indicated, TNC complexes were enriched from enzyme-digested thymic cell suspensions by four-step 1×g sedimentation in fetal bovine serum (FBS) as described ^{(Wekerle et al., 1980)}. Single cell suspension was obtained from the enriched TNC samples by gentle mechanical dissociation of the complexes with a 3 ml syringe and 29 G needle ^{(Nakagawa et al., 2012)}.

### 6.4 Preparation of Blood Cell Suspension

For peripheral blood analysis, EDTA was used as an anticoagulant reagent and added at a concentration of 1.5 mg per ml of blood. The erythrocyte-lysing buffer (555899; BD Biosciences) was used to prepare blood sample for flow cytometric analysis and cell sorting. The mixed panel of BV421-conjugated anti-CD3, PE-CF594-conjugated anti-CD4 and Alexa Fluor 647-conjugated anti-CD8 was used to examine or sort T-helper, T-cytotoxic and immature CD4⁺CD8⁺ cells.

6.5 Preparation of Lymphocyte Suspension from Liver

After perfusion with PBS, liver tissue was homogenized through a 100 μm cell straner and centrifuged at 1600 rpm for 5 min, then discard the supernatant. Pellets were resuspended and digested in DMEM containing 0.5 mg/ml collagenase Type IV (Gibico™) and 150 m/ml DNase I (Sigma-Aldrich) at 37° C. for 40 minutes. Digestion buffer were centrifuged at 1600 rpm for 5 min to collect cell pellets. Cell pellets were washed and resuspended with PBS. Cell suspensions were mounted on a gradient containing 40% and 70% Percoll (GE Healthcare Bio-Sciences, Sweden) for centrifugation at 1126×g for 20 min at 4° C. The middle layers between 40% and 70% Percoll were collected for subsequent analyses.

### 6.6 Antibody Labeling

Antibodies were obtained from Biolegend (San Diego, Calif., USA), BD Bioscience, eBioscience (San Diego, Calif., USA) or Vector (Burlingame, Calif., USA). For all staining, Fc receptors were blocked with anti-CD16/32 (eBioscience 14-0161-82) before antibody labeling. After centrifugation at 400×g, cells were incubated with specific combinations of antibodies on ice for 30 minutes. The mixed panel of Pacific Blue™-conjugated anti-CD3 (Biolegend 100214), phycoerythrin (PE)-CF594-conjugated anti-CD4 (BD 562285), Alexa Fluor® 647-conjugated anti-CD8a (Biolegend 100724), fluorescein isothiocyanate (FITC)-conjugated anti-CD44 (BD 553133) and PE-Cy™ 7-conjugated anti-CD25 (BD 552880) was used for analyzing CD4⁻CD8⁻ double-negative (DN), CD4⁺CD8⁺ double-positive (DP), CD3⁺CD4⁺ or CD3⁺CD8⁺ single-positive (SP) cells. The mixed panel of BD Horizon™ V450-conjugated anti-lineage cocktail (561301; BD Biosciences) including 500A2 recognizing mouse CD3e, M1/70 recognizing CD11b; RA3-6B2 recognizing CD45R/B220, TER-119 recognizing Ly-76 mouse erythroid cells, and RB6-8C5 recognizing Ly-6G and Ly-6C was used for cell depletion. FITC-conjugated anti-CD44, PE-Cy7-conjugated anti-CD25, allophycocyanin (APC)-conjugated anti-c-Kit (Biolegend 135108) and PE-conjugated anti-CD24 (BD 553262) was used to examine DN1-4 populations and early thymic progenitors (ETPs). The mixed panel of PE-conjugated anti-CD45 (Biolegend 103106), APC-conjugated anti-EpCAM (Biolegend 118214), PE-Cy7-conjugated anti-Ly51 (Biolegend 108314) and FITC-conjugated anti-UEA-I (Vector FL-1061) was used to evaluate the cortical (cTEC) and medullary (mTEC) thymic epithelial cells. For analyzing samples from Adn-Cre/ROSA^{mT/mG} or Adn-Cre/ROSA^{mT/mG}-AKO mice, Pacific Blue™-conjugated anti-CD45 (Biolegend 103125), anti-CD4 (Biolegend 100427), anti-CD25 (Biolegend 102021) were used to avoid the interference from cells with green or red fluorescence. Fixed and permeabilized lymphocytes from liver were stained with PE-conjugated anti-IL17A (Biolegend 506903), BV421-conjugated anti-CD45 (Biolegend 103134) and APC-conjugated anti-CD4 (Biolegend 100412) to detect Th17 cells.

### 6.7 Procedure for Analyzing TNC Complexes

Freshly collected thymus tissues were cut, minced and transferred to DMEM containing 2 mg/ml collagenase Type I and 40 μg/ml DNase I. After incubation at 37° C. with shaking for 30 minutes, albumin-rich buffer was added to stop digestion. Cells were then strained through ac100 μm filter mesh and centrifuged at 400×g. The pellets were washed and re-suspended in PBS for flow cytometric analyses. Briefly, the thymic cell suspensions were stained with surface antibodies including BV421-conjugated anti-CD45 (eCD45) and APC-conjugated anti-EpCAM (CD326) in the dark on ice for 30 minutes, then fixed and permeabilized with Cytofix/Cytoperm™ Fixation/Permeabilization Kit (BD Biosciences, 554714). The cells were subsequently incubated with antibodies including PE-conjugated anti-CD45 (iCD45) and anti-β5t antibodies (MBL Life science PD021) followed by Alexa Fluor 488-conjugated anti-rabbit IgG antibody (Abcam ab150077) in the dark on ice for another 30 minutes. Where indicated, cells were stained with 10 m/mL PI after fixation and permeabilization.

### 6.8 Confocal Live Cell Imaging

Enriched TNC samples from Adn-Cre/ROSA^{mT/mG} or Adn-Cre/ROSA^{mT/mG}-AKO mice were prepared in a temperature-controlled (37° C.) and pH-stable chamber for 3D live cell imaging using the UltraVIEW® VOX Spinning Disc confocal system (Perkin Elmer). Five percent CO₂ was continuously supplied during the experiment. Images were acquired every 15 minutes by a confocal fluorescent microscopy (Inverted: Nikon Eclipse Ti-E) with a mecury/xenon/LED's lamp. 0.1% stock concentration 4′,6-diamidino-2-phenylindole (DAPI, 20 mg/mL, Sigma-Aldrich D9542, US) was added into the chamber before image acquisition Volocity® acquisition, visualization and quantification software (Perkin Elmer) was used for image analyses. Both Max intensity and Iso surface mode were used to display the dynamic video.

### 6.9 In Situ Hybridization

Thymic cell suspensions were plated onto the slides using a Cytopro™ centrifuge (Wescor, Utah, USA). In situ hybridization was performed using the RNAscope® Intro Pack 2.5 HD Reagent Kit BROWN-Mm (Cat No 322371; Advanced Cell Diagnostics, Beijing, China), which contained eleven pairs of double “Z” oligo probes (RNAscope® Probe-Mm-Adipoq; Cat No. 440051) to target the 1-640 bp of murine Adipoq (NCBI Reference Sequence: NM_009605.5). The hybridization signals were developed by applying the RNAscope® DAB reagent (Advanced Cell Diagnostics). All sections were counterstained with hematoxylin before mounting on glass coverslips with a xylene based mounting medium.

### 6.10 Reverse Transcription (RT-PCR) and Quantitative PCR (QPCR)

RNAiso Plus (TaKaRa, 9108/9109) was used to isolate the total RNA from tissue or cell samples. After checking the quality by 2100 bioanalyzer (Agilent, Santa Clara, Calif., USA), reverse transcription was performed using PrimeScript™ RT reagent Kit (TaKaRa). Reverse transcription PCR (RT-PCR) was performed for amplifying the Adipoq transcript and the products analyzed by agarose electrophoresis. Quantitative PCR (QPCR) was performed using SYBR® Green real-time PCR reagents from Qiagen (Hilden, Germany). The reactions were carried out in a 7000 Sequence Detection System (Applied Biosystems, Foster City, Calif., USA). Quantification was achieved by comparing the Ct values that were normalized with 18S rRNA or β-actin as the internal controls. The QPCR primers are listed in Table 2.

TABLE 2
List of primers used for RT-PCR and QPCR.

| Gene | Experiment | Primer sequences | Product |
|---|---|---|---|
| Adipoq | RT-PCR | (F) 5′-AAGCTCTCCTGTTCCTCT | 720 bp |
| | | TAATC-3′ | |
| | | (R) 5′-CATGGTAGAGAAGAAAGC | |
| | | CAGTA-3′ | |
| Adipoq | RT-PCR | (F) 5′-TGCCGAAGATGACGTTAC | 660 bp |
| | | TAC-3′ | |
| | | (R) 5′-GTAGAGTCGTTGACGTTA | |
| | | TCT-3′ | |
| Adipoq | QPCR | (F) 5′-CTCCACCCAAGGGAACTT | 141 bp |
| | | GT-3′ | |
| | | (R) 5′-TAGGACCAAGAAGACCTG | |
| | | CATC-3′ | |
| FGF21 | QPCR | (F) 5′-TATGGATCGCCTCACTTT | 123 bp |
| | | GA-3′ | |
| | | (R) 5′-GGAGTCCTTCTGAGGCAG | |
| | | AC-3′ | |
| PPARα | QPCR | (F) 5′-GAGTGCAGCCTCAGCCAA | 121 bp |
| | | GTTGA-3′ | |
| | | (R) 5′-AGGCAGGCCACAGAGCGC | |
| | | TAA-3′ | |
| TNFα | QPCR | (F) 5′-TCGTAGCAAACCACCAAG | 207 bp |
| | | TG-3′ | |
| | | (R) 5′-AGATAGCAAATCGGCTGA | |
| | | CG-3′ | |
| MCP-1 | QPCR | (F) 5′-TGATCCCAATGAGTAGGC | 132 bp |
| | | TGGAG-3′ | |
| | | (R) 5′-ATGTCTGGACCCATTCCT | |
| | | TCTTG-3′ | |
| TGFβ | QPCR | (F) 5′-TGGAGCAACATGTGGAAC | 108 bp |
| | | TC-3′ | |
| | | (R) 5′-CGTCAAAAGACAGCCACT | |
| | | CA-3′ | |
| IL-6 | QPCR | (F) 5′-TGGAGTCACAGAAGGAGT | 155 bp |
| | | GGCTAAG-3 | |
| | | (R) 5′-TCTGACCACAGTGAGGAA | |
| | | TGTCCAC-3′ | |
| VEGFα | QPCR | (F) 5′-AACGATGAAGCCCTGGAG | 120 bp |
| | | TG-3′ | |
| | | (R) 5′-TGAGAGGTCTGGTTCCCG | |
| | | A-3′ | |

TABLE 3
List of antibodies used for immunocytochemistry (ICC), immunohistochemistry
(IHC) and Western blotting (WB) experiment.

| | | Catalog | |
|---|---|---|---|
| Antibody (species) | Company | number | Dilution (application) |
| Anti-murine adiponectin | Immunodiagnostics, Hong | 12010 | 1:3000 (WB), 1:1000 |
| (rabbit) | Kong, China | | (IF) |
| Anti-cytokeratin 5 (goat) | Santa Cruz Biotechnology, | sc-17090 | 1:50 (IF) |
| | Santa cruz, CA | | |
| Anti-cytokeratin 8 (goat) | Santa Cruz Biotechnology | sc-241376 | 1:50 (IF) |
| Anti-neuropilin-1 (goat) | R&D system, Inc., | AF566 | 5 μg/ml (IF) |
| | Minneapolis, MN, USA | | |
| Anti-CD31 (goat) | R&D system, Inc., | AF3628 | 1:200 (IF) |
| | Minneapolis, MN, USA | | |
| Anti-galectin-3 (rat) | Santa Cruz Biotechnology | sc-23938 | 1:200 (WB), 1:100 (IF) |
| Anti-β5t (rabbit) | MBL Life science, | PD021 | 1:200 (IF) |
| | Nagoya, Japan | | |
| Anti-β-actin (mouse) | Sigma-Aldrich, St Louis, | A1978 | 1:1000 (WB) |
| | MI, USA | | |
| Anti-Neuropilin-1 | R&D, Minneapolis, MN, | AF566 | 1:300 (IF) |
| (mouse) | U.S.A. | | |
| Anti-CD72 (mouse) | R&D, Minneapolis, MN, | AF1279 | 1:300 (IF) |
| | U.S.A. | | |
| Anti-CD100 (mouse) | Abcam, Cambridge, U.K. | Ab231961 | 1 μg/ml (WB), 5 μg/ml |
| | | | (IF) |
| Anti-CD100 (human) | LS-Bio, Seattle, WA, | LS-B12098- | 1:2000 (WB) |
| | U.S.A. | 50 | |

### 6.8 Evaluation of Metabolic Function

Body weight and fat mass composition were measured between 1000 and 1200 h for mice that were either starved overnight or fed ad libitum. The body mass composition was assessed in conscious and unanesthetized mice using a Bruker minispec Body Composition Analyzer (Bruker Optics, Inc., Woodlands, Tex.). Blood glucose was monitored by tail nicking using an Accu-Check Advantage II Glucometer (Roche Diagnostics, Mannheim, Germany). The intraperitoneal glucose tolerance test (ipGTT) and insulin tolerance test (ITT) were performed as previously described (Xu et al., 2015). Circulating and tissue contents of lipids, including triglycerides, total cholesterols, and free fatty acids, were analyzed using LiquiColor Triglycerides and Stanbio Cholesterol (Stanbio Laboratory, Boerne, Tex.) and the Half-Micro Test Kit (Roche Diagnostics), respectively. The fasting serum insulin concentration was quantified using the commercial ELISA kits from Mercodia AB (Uppsala, Sweden). Metabolic rate (VO2, VCO2, and respiratory exchange ratio [RER]) was measured by indirect calorimetry using a six-chamber open-circuit Oxymax system component of the Comprehensive Laboratory Animal Monitoring System (CLAMS; Columbus Instruments, Columbus, Ohio) as previously described (14). All mice were acclimatized to the cage for 48 h before recording the parameters.

### 6.9 Data Analysis

All experiments were performed with six to eight samples per group and results derived from at least three independent measurements. Values are expressed as mean±SEM. Statistical calculations were done using the Statistical Package for the Social Sciences version 11.5 software package (SPSS, Inc., Chicago, Ill., USA). The statistical analyses were performed using SPSS Statistics software 22.0 (IBM, Armonk, N.Y., USA). Comparison between groups was performed using Student's unpaired t-test or two-way ANOVA (GraphPad Prism 7.00 Software, Inc., San Diego, Calif., USA). P values less than 0.05 were accepted to indicate statistically significant differences.

### 7. RESULTS

### 7.1 Adiponectin-Expressing Cells in Thymus

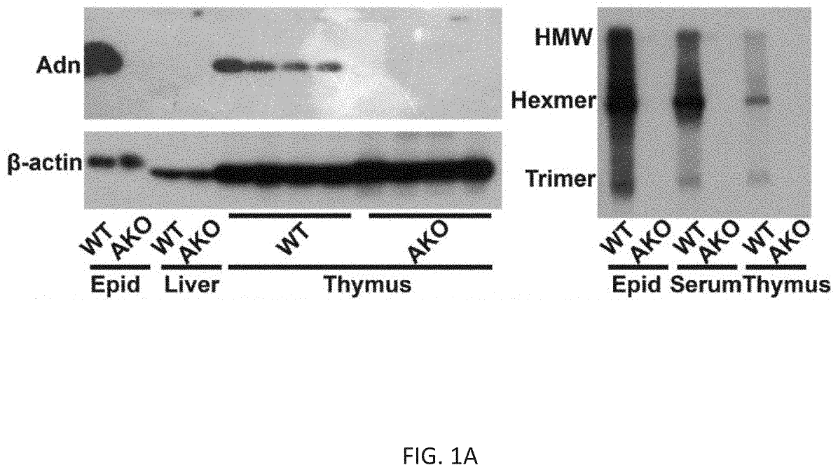

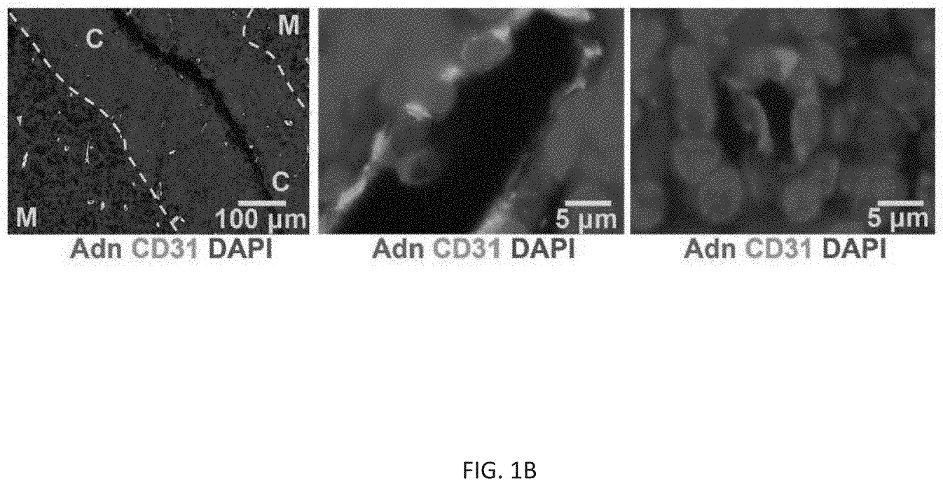

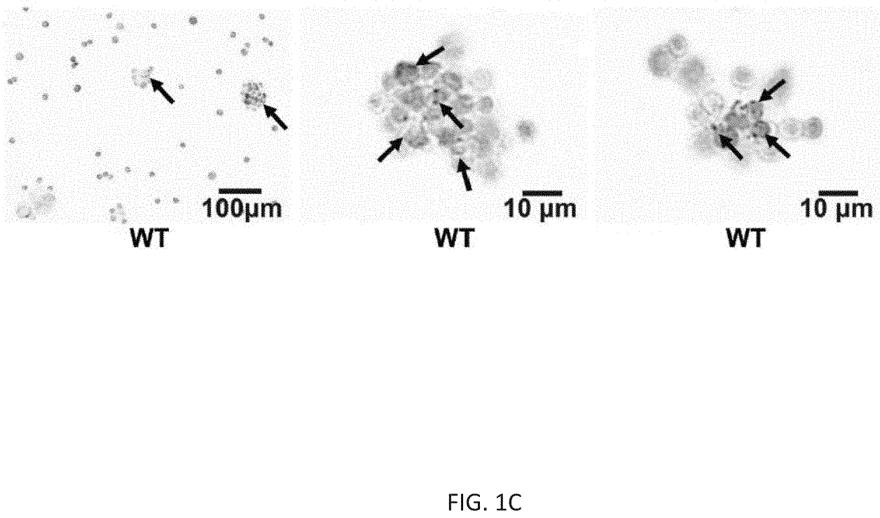

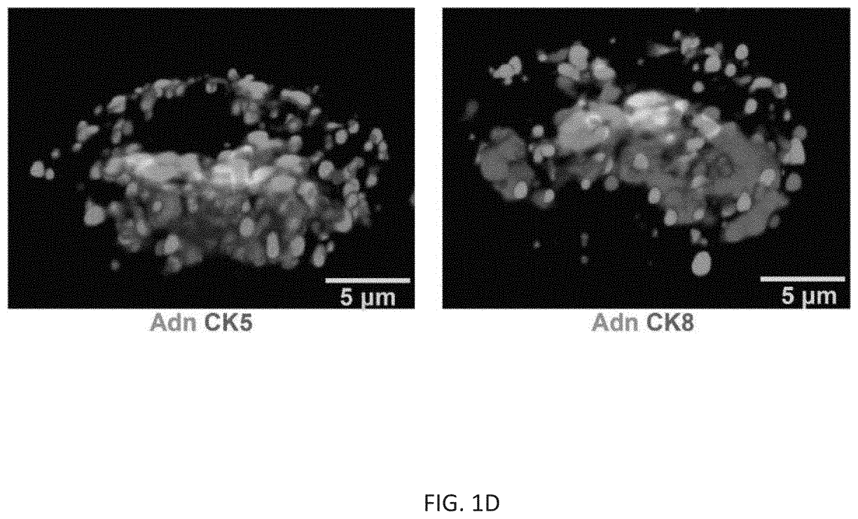

Adiponectin protein was detected in the thymus of wild type (WT) mice and existed as trimer, hexamer and high molecular weight (HMW) oligomers (FIG. 1A). The protein concentration of adiponectin was 1.2576±0.1417 μg/mg and 0.0065±0.0015 μg/mg in epididymal adipose and thymus tissues, respectively, as measured by an in-house ELISA. Immunofluorescence analyses revealed that adiponectin protein was present across the outer cortex, the cortico-medullar and medullar regions, but not co-localized with that of CD31, which labels endothelial cells of arteries, veins and capillaries (FIG. 1B).

The full length mRNA transcript of Adipoq was detected in both epididymal adipose tissue and thymus (Supplementary FIG. 1A). In situ hybridization revealed that most of the cells containing the mRNA transcript of Adipoq were located within the lymphoepithelial cell clusters (FIG. 1C), which were positively stained with antibodies against cytokeratin 5 and/or cytokeratin 8 (FIG. 1D), markers of the thymic nurse cell (TNC) complexes ^{(Hendrix et al., 2010)}. In contrast to the Adipoq transcript, which was present in only a few individual cells (FIG. 1C), adiponectin protein was widely distributed at the extracellular space within the TNC complexes (FIG. 1D).

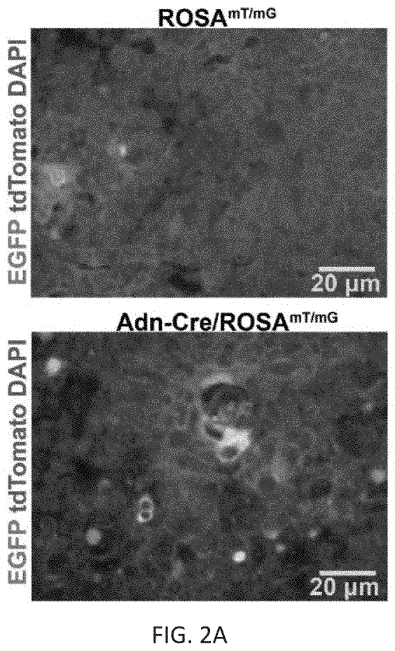

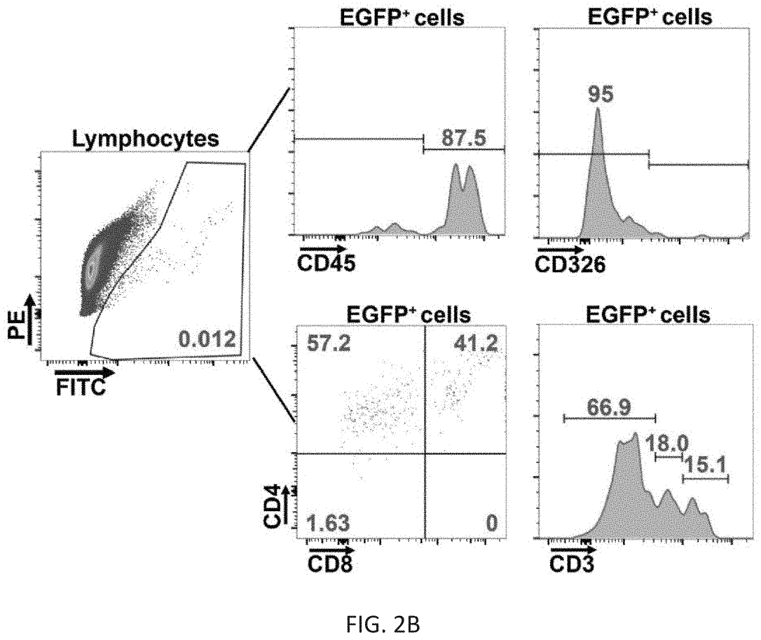

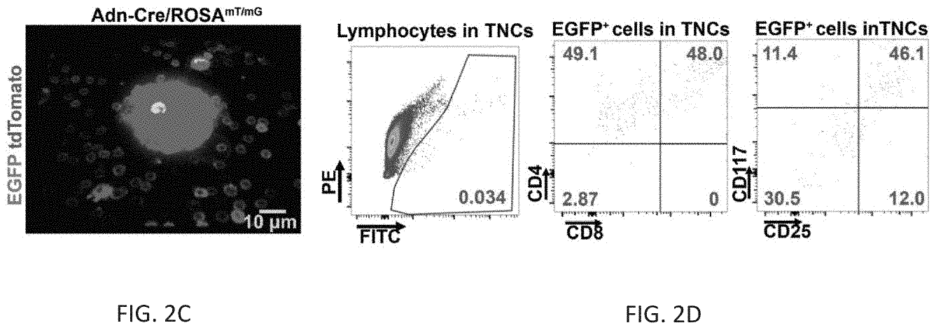

In thymus of the transgenic two-color reporter mouse model (Adn-Cre/ROSA^{mT/mG}), the expression of Cre recombinase driven by the Adipoq promoter resulted in permanent, stable, and highly specific EGFP (mG) signals, replacing the cell membrane-localized tdTomato (mT) fluorescence in the thymus tissue (FIG. 2A). Flow cytometry was performed to examine the nature of adiponectin-expressing cells in the thymus of Adn-Cre/ROSA^{mT/mG} mice. Around 0.012% of the total thymic populations were EGFP⁺ cells, which were labelled positively with CD45 [a lymphohematopoietic surface antigen] and negatively with CD326 [an epithelial adhesion molecule], distributed in CD4⁺ single positive (SP) and CD4⁺CD8⁺ double positive (DP) subpopulations, with no, low or high CD3 expressions (FIG. 2B).

In cell suspension prepared from the thymus of Adn-Cre/ROSA^{mT/mG} mice, most of the EGFP⁺ cells resided within the lymphoepithelial TNC complexes (FIG. 2C), where many lymphocytes labelled with mT fluorescence were actively engulfed and released (Supplementary Online Video 1). In samples containing enriched TNC complexes, the amount of Adipoq mRNA transcript was ˜four-fold higher than that of the thymus tissue of Adn-Cre/ROSA^{mT/mG} mice (Supplementary FIGS. 1, B and C). Approximately 0.034% of the total number of thymocytes within TNC complexes were EGFP⁺ and distributed in CD4⁺ SP and CD4+CD8⁺ DP subpopulations (FIG. 2D). Moreover, about half of the EGFP⁺ within the TNC complexes were positively labelled with markers of the canonical T-lineage precursors, CD117/cKit [the stem cell factor receptor] and CD25 [the α chain of the IL-2 receptor]^{(Perrin et al., 2004)}.

7.2 Adiponectin-Expressing Thymic Regulatory T-Cells (tTreg)

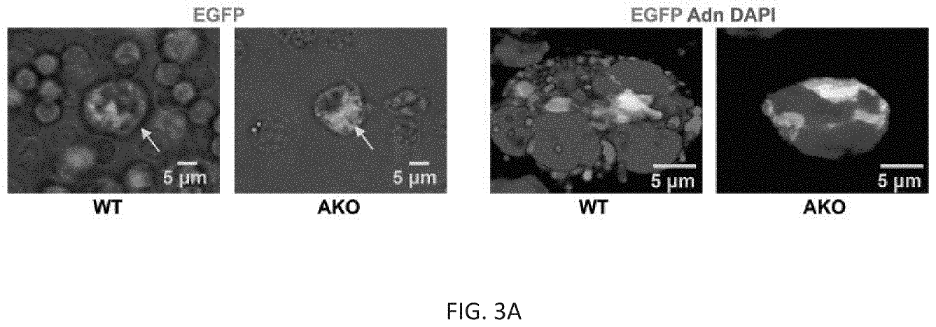

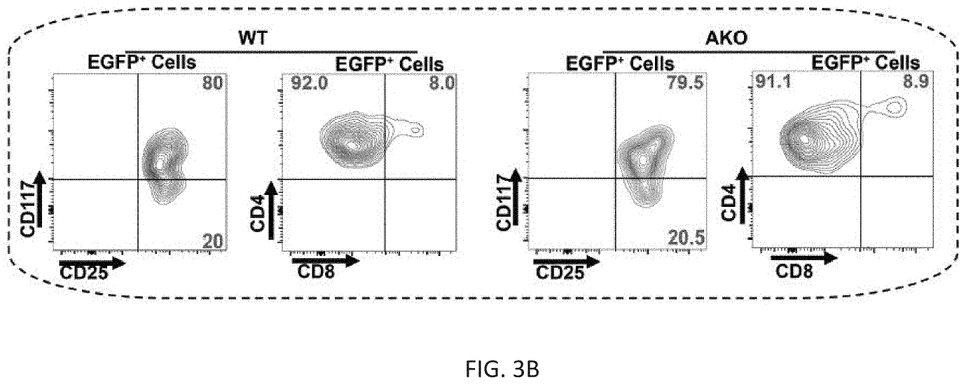

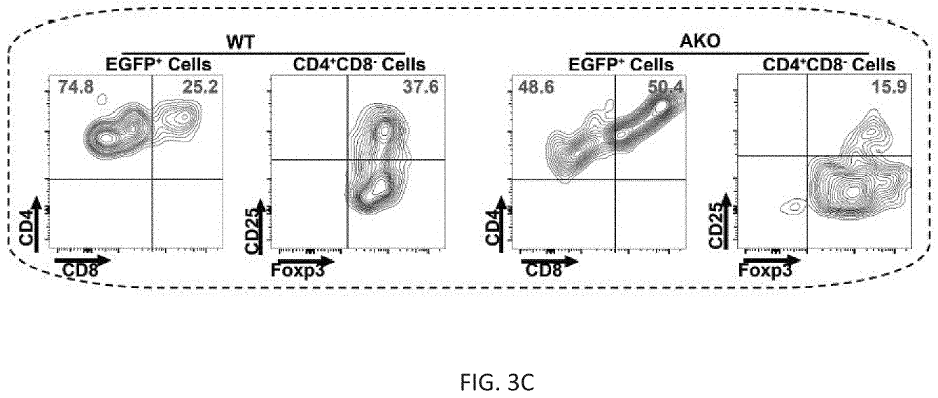

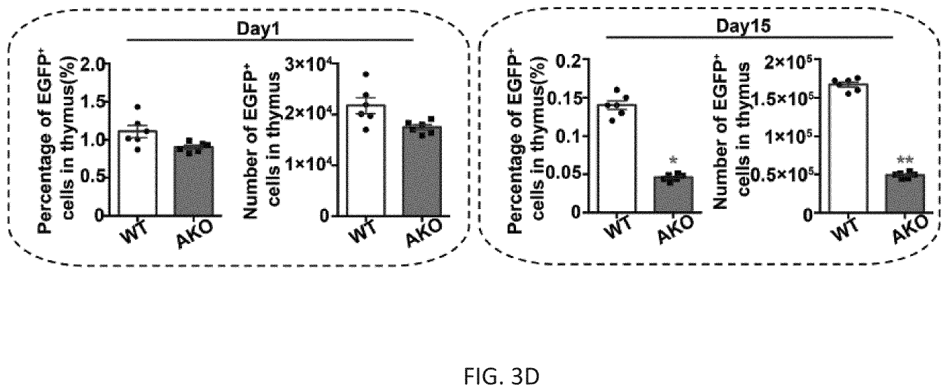

Adiponectin-expressing EGFP⁺ cells collected from the thymus of five-weeks old Adn-Cre/ROSA^{mT/mG} mice were adoptively transferred (30000 EGFP⁺ cells/mouse by tail vein injection) into sub-lethally irradiated WT or adiponectin knockout (AKO) mice. Thymus, spleen, liver and adipose tissues were collected from the recipient mice at one-day and 15-days after tail vein injection for subsequent analyses. Within 12-hours after the injection, the majority of EGFP⁺ cells were present within the TNC complexes isolated from thymus tissues of both WT and AKO recipient mice (FIG. 3A, left). There were barely any EGFP⁺ cells in other tissues including spleen, liver and epididymal fat pads after the adoptive transfer (data not shown). In TNC complexes of WT recipient mice, adiponectin protein was distributed around many of the engulfed thymocytes, including the EGFP+ cells. By contrast, only few adiponectin protein signals were located in close proximity to the EGFP+ cells within the TNC complexes of AKO recipient animals (FIG. 3A, right).

The lineage (Lin)-negative thymocytes were collected from the thymus of WT and AKO recipient mice for flow cytometric analyses of donor-derived EGFP⁺ cells. On the first day after injection, the majority of EGFP⁺ were CD117+CD25+ and CD4+, with less than 10% exhibiting CD4⁺CD8⁺ in the thymus of both WT and AKO recipient mice (FIG. 3B). On the 15^{th} day after injection, approximately 25% and 50% of EGFP⁺ cells were CD4⁺CD8⁺ in WT and AKO thymus, respectively (FIG. 3C). Note that over 30% of EGFP⁺ cells in WT thymus exhibited CD4⁺CD25⁺Foxp3⁺ (FIG. 3C), the characteristic feature of regulatory T-cells (Treg) ^{(Rodriguez-Perea et al., 2016)}. Compared to WT samples, the percentage content as well as the total number of EGFP⁺ cells in the thymus of AKO were significantly decreased at day 15 after the adoptive transfer (FIG. 3D). As a result, the total number of the mature EGFP⁺ Treg in AKO thymus was significantly less than those of WT recipient mice (3790±285 and 46968±2225, P<0.05).

In TNC complexes of Adn-Cre/ROSA^{mT/mG} mice, over 10% of EGFP⁺ cells were CD4⁺CD25⁺Foxp3⁺. In TNC samples of WT mice adoptively transferred with EGFP⁺ thymocytes collected from the thymus of Adn-Cre/ROSA^{mT/mG} mice, the adiponectin-expressing cells were in close contact with neuropilin-1 (Nrp1) protein signals, but located away from the area with positive galectin-3 staining (Supplementary FIG. 2A). In TNC samples of AKO mice adoptively transferred with the EGFP⁺ thymocytes collected from the thymus of Adn-Cre/ROSA^{mT/mG} mice, however, the majority of adiponectin-expressing cells were not interacting with Nrp1 protein signals but in close contact with galectin-3 (Supplementary FIG. 2B). Consistently, the distribution of adiponectin protein was intercalated with that of Nrp1, but different from that of galectin-3 in TNC complexes of WT mice (Supplementary FIG. 2C).

The above results suggest that adiponectin-expressing cells are thymic Treg (tTreg) precursors, characterized by high expression of CD117 as well as CD4⁺CD25⁺CD8⁻, and developed into mature tTreg primarily within the TNC complexes.

7.3 Insulin-Sensitizing Activity of the Adiponectin-Expressing tTreg Precursors

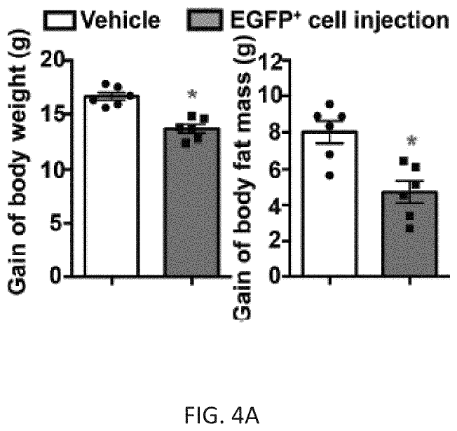

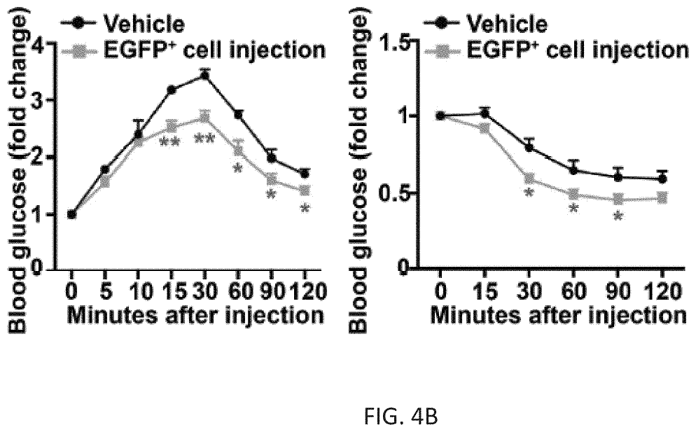

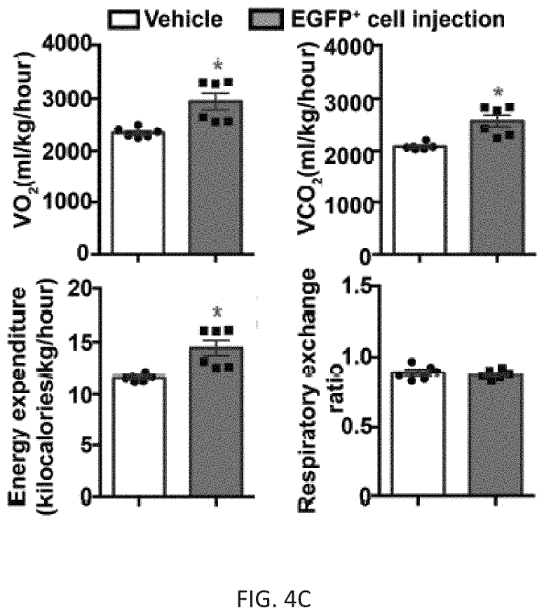

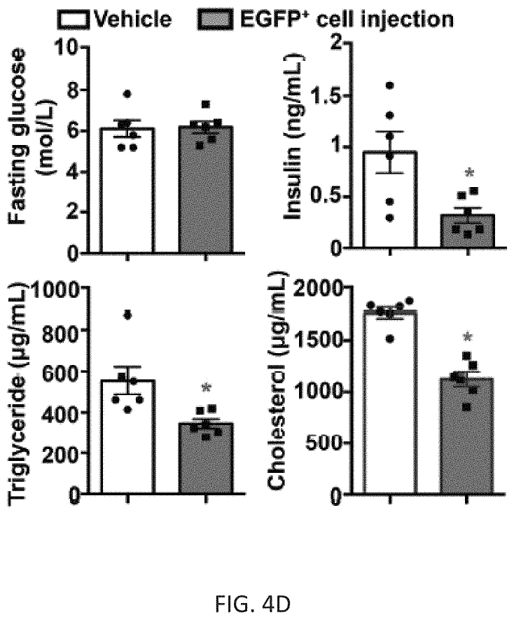

Adoptive transfer of the adiponectin-expressing EGFP⁺ cells was performed in four-weeks old WT mice (30000 cells/mouse by tail vein injection). After 18-weeks of high fat diet (HFD) feeding, the gains of body weight and body fat percentage were significantly less in EGFP⁺ cell-treated mice than those of vehicle controls (FIG. 4A). Mice treated with adiponectin expressing EGFP⁺ cells exhibited significantly improved glucose tolerance and insulin sensitivity (FIG. 4B), enhanced oxygen consumption (VO₂), CO₂ production (VCO₂) and energy expenditure, but similar respiratory exchange ratio (RER) when compared to those of vehicle control group (FIG. 4C). While there was no significant difference in fasting glucose levels, the plasma concentrations of insulin, triglyceride and cholesterol were all significantly reduced in mice treated with adiponectin expressing EGFP⁺ cells (FIG. 4D). Moreover, the circulating liver injury markers, including alanine (ALT) and aspartate (AST) transaminases, were significantly decreased in mice treated with adiponectin expressing EGFP⁺ cells (185.01±75.71 and 118.08±42.87 U/L, respectively), when compared to the vehicle control group (300.28±74.01 and 199.20±69.64 U/L, respectively).

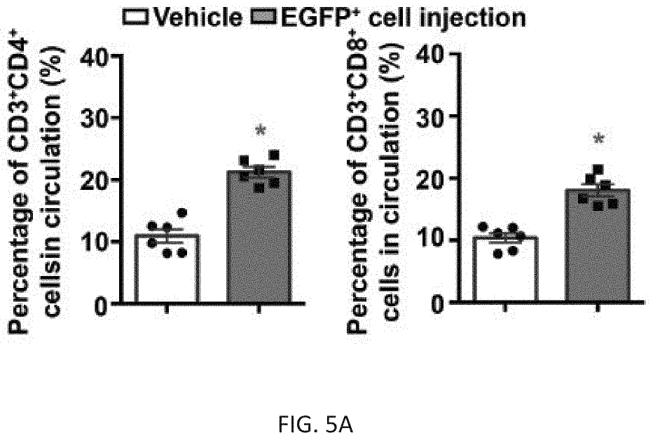

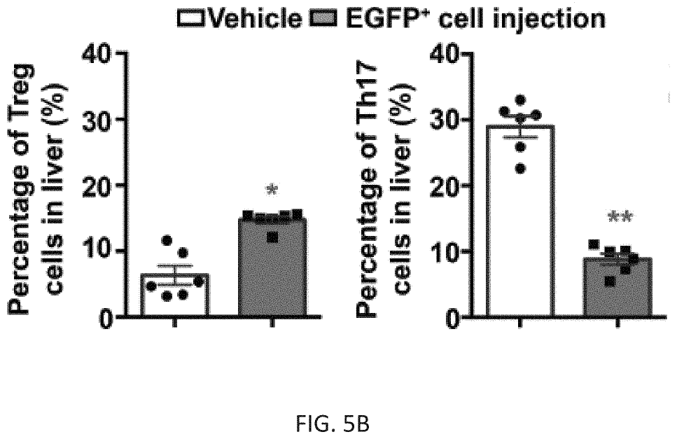

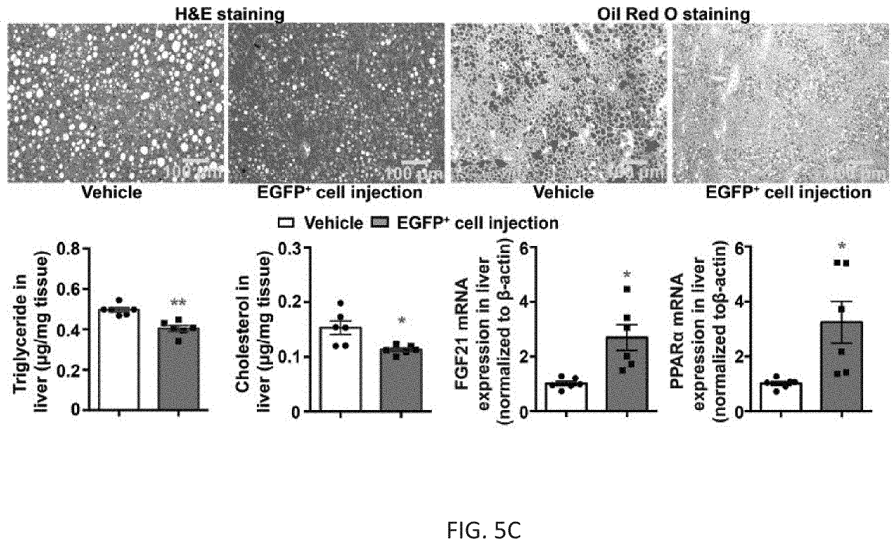

In blood, mice treated with adiponectin-expressing EGFP⁺ cells contained significantly increased amounts of circulating CD3⁺CD4⁺ and CD3⁺CD8⁺ T-cells (FIG. 5A). In liver, the percentage number of Treg significantly increased, whereas that of Th17 cells significantly decreased in mice treated with adiponectin-expressing EGFP+ cells (FIG. 5B). Note that the EGFP⁺ cells characterized by CD4⁺CD8⁻CD25⁺Foxp3⁺ and positive staining with Nrp1 were present in livers of mice treated with adiponectin-expressing tTreg precursors (Supplementary FIG. 3). HFD-induced hepatic steatosis was significantly attenuated by the treatment with adiponectin-expressing tTreg precursors, as demonstrated by histological imaging analyses, tissue lipid contents and mRNA expression levels of peroxisome proliferator-activated receptor alpha (PPARa) and fibroblast growth factor 21 (FGF21) (FIG. 5C). In epididymal adipose tissue, treatment with adiponectin-expressing EGFP+ cells reduced the average adipocyte size and the mRNA expression of inflammatory markers, including monocyte chemoattractant protein-1 (MCP-1) and tumor necrosis factor alpha (TNFα), but significantly increased the Adipoq transcript levels (FIG. 5D).

Collectively, the data demonstrate that adiponectin-expressing tTreg precursors elicit potent insulin-sensitizing and hepatoprotective activity via regulating T-cell homeostasis in the circulation and the immune microenvironment in peripheral organs.

7.4 Anti-Breast Cancer Activity of the Adiponectin-Expressing tTreg Precursors

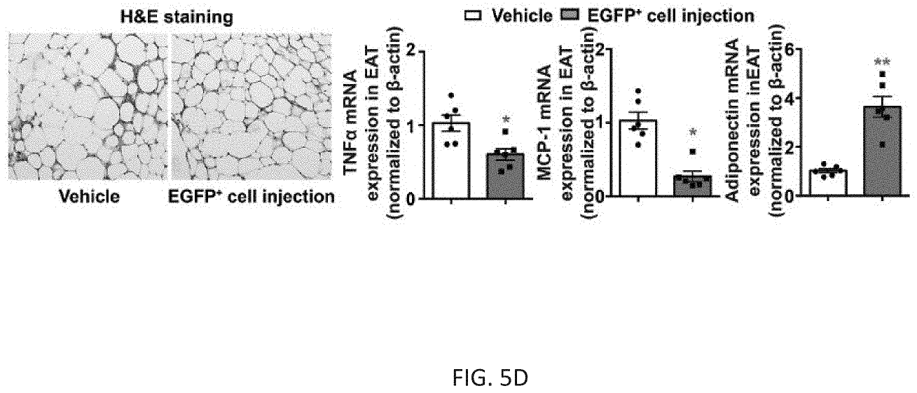

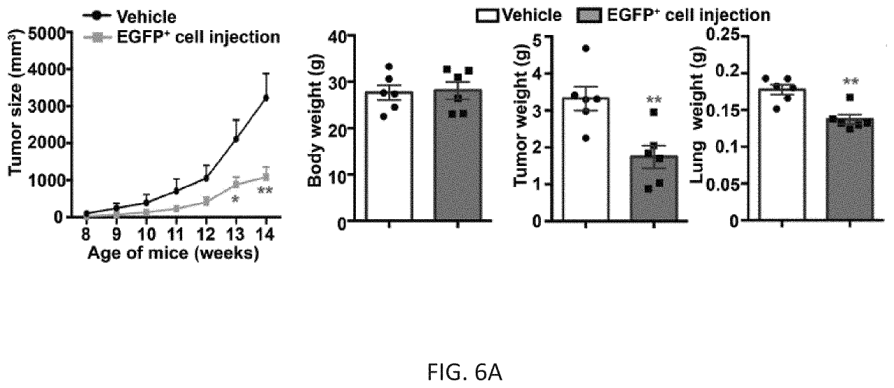

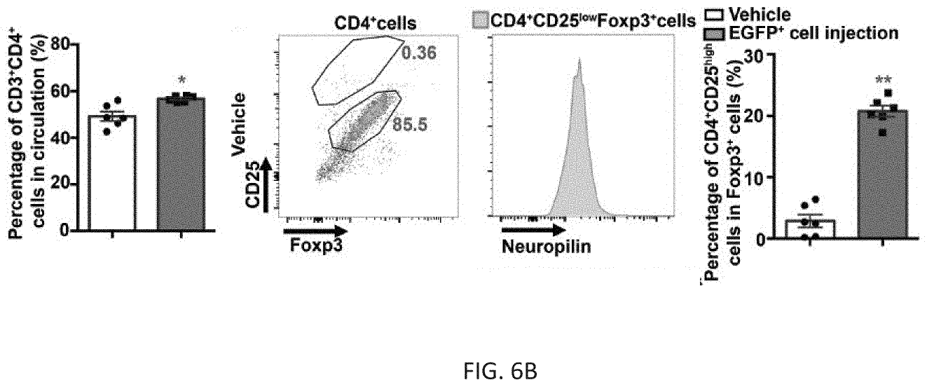

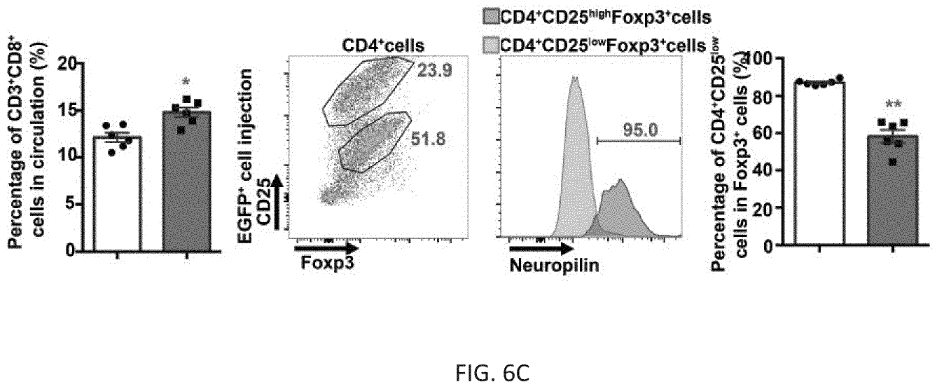

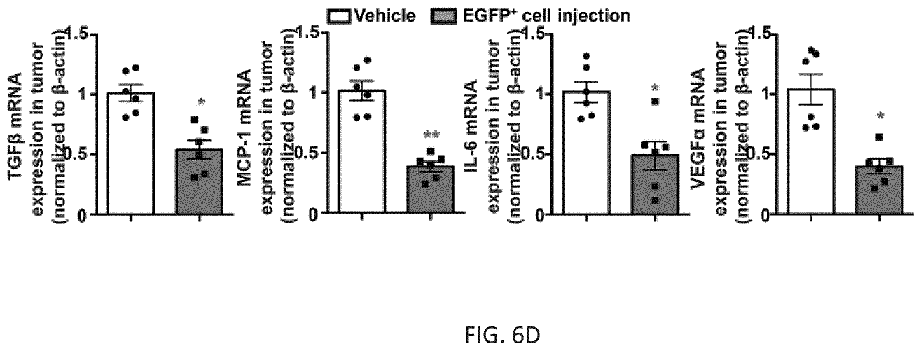

The anti-tumor activity of adiponectin-expressing EGFP⁺ cells was evaluated in female MMTV-PyVT mice that develop aggressive mammary tumors from the age of seven- or eight-weeks ^{(Lam et al., 2009)}. Adoptive transfer was performed in four-weeks old MMTV-eight-weeks PyVT animals (30000 cells/mouse) by intravenous injection of EGFP⁺ thymocytes collected from the thymus of Adn-Cre/ROSA^{mT/mG} mice. Mammary tumor development was monitored every week until the age of 14-weeks. Compared to vehicle controls, treatment with adiponectin-expressing EGFP⁺ cells significantly inhibited the development of mammary tumors (FIG. 6A). When compared to vehicle controls, the weights of tumor were reduced by over ˜45% and those of the lung decreased by ˜25% in MMTV-PyVT mice treated with adiponectin-expressing EGFP⁺ thymocytes (FIG. 6A).

Flow cytometry was performed to analyze the composition of T lymphocyte subsets. In blood samples collected from 14-weeks old MMTV-PyVT mice, the total amount of CD3⁺CD4⁺ and CD3⁺CD8⁺ was both significantly increased by the treatment with the adiponectin-expressing EGFP⁺ thymocytes (FIG. 6B). Moreover, a distinct population of Nrp1⁺ Treg cells, characterized by CD4⁺CD25^{high}Foxp3⁺ were present in mammary tumors of MMTV-PyVT mice treated with adiponectin-expressing EGFP⁺ cells (FIG. 6C). Compared to those of the vehicle group, the mRNA expression levels of transforming growth factor beta (TGFβ), monocyte chemoattractant protein-1 (MCP1), interleukin-6 (IL6) and vascular endothelial growth factor alpha (VEGFα) were significantly downregulated in tumor samples of MMTV-PyVT mice treated with adiponectin-expressing EGFP⁺ cells (FIG. 6D).

Compared to MMTV-PyVT mice containing wild type Adipoq alleles (PyVT-WT), those lacking adiponectin expression (PyVT-AKO) were not responsive to the same treatment with EGFP⁺ thymocytes collected from the thymus of Adn-Cre/ROSA^{mT/mG} mice (data not shown). In PyVT-AKO mice, the CD3⁺CD4⁺ T-cells were significantly reduced, accompanied by an elevated number of immature CD3⁻CD4⁺CD8⁺ cells in the blood circulation (Supplementary FIG. 4A). The latter population of cells promoted mammary tumor development in NOD/SCID mice implanted with human breast cancer MDA-MB-231 cells (Supplementary FIG. 4B).

In summary, the results suggest that adiponectin-expressing tTreg precursors exert anti-breast cancer activity at least partly via modulating the repertoire of T-cells in the blood circulation as well as mammary tumor tissues.

7.5 Adiponectin Facilitates T-Cell Selection within the TNC Complexes

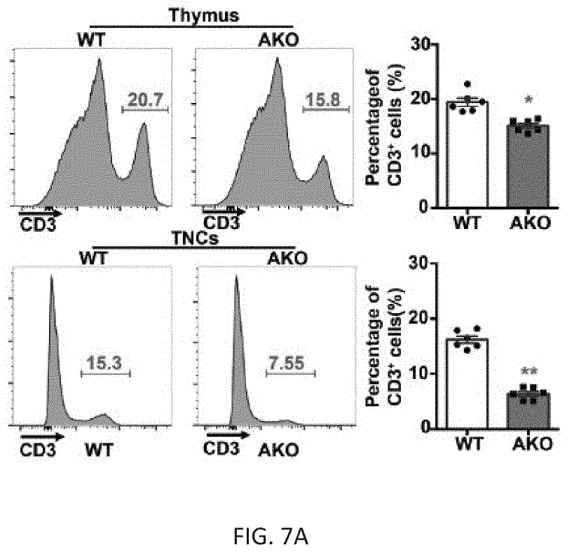

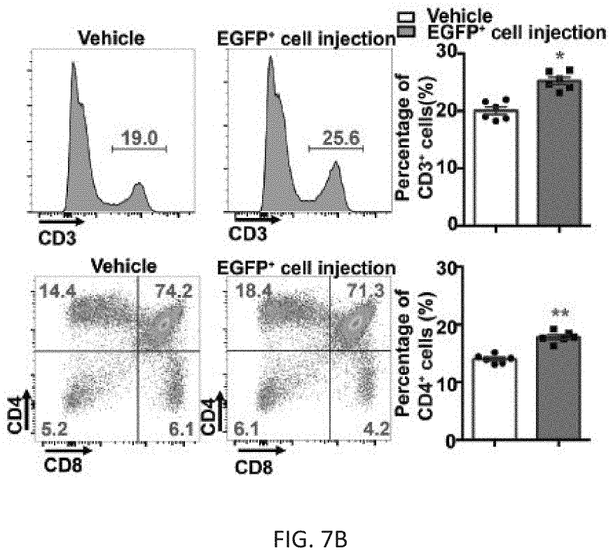

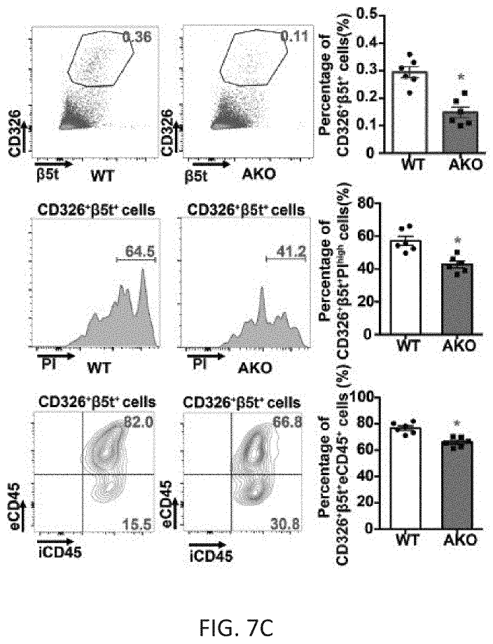

Flow cytometry was performed to evaluate the CD3⁺ thymocytes in the thymus and the enriched TNC samples collected from seven-weeks old WT and AKO mice. Compared to those of WT animals, the percentage contents of CD3⁺ cells were significantly reduced in both thymus and TNC complexes of AKO mice (FIG. 7A). In the thymus of WT mice treated with adiponectin-expressing tTreg precursors, the amount of CD45⁺CD3⁺ and CD4⁺ SP cells significantly increased when compared to the vehicle control animals (FIG. 7B). The number of TNC complexes, characterized by CD326⁺β5t⁺PI^{high (Nakagawa et al., 2012)}, was significantly decreased in the thymus of AKO mice when compared to WT animals (FIG. 7C). Moreover, the two-color staining of CD45 for extracellular (eCD45) and intracellular (iCD45) thymocytes revealed that the amount of thymocytes attached to TNC complexes was significantly less in preparations from AKO mice than those of WT samples (FIG. 7C).

Live cell imaging was performed with TNC complexes isolated from Adn-Cre/ROSA^{mT/mG} mice lacking the Adipoq alleles (Adn-Cre/ROSA^{mT/mG}-AKO). There were few cells labelled with mT fluorescence released from the TNC complexes, which did contain the EGFP⁺ cells (Supplementary Online Video 2). With time, the number of apoptotic cells within the TNC complexes that were actively uptaking the diamidino-2-phenylindole (DAPI) DNA-specific dye was significantly less than those isolated from Adn-Cre/ROSA^{mT/mG} mice (Supplementary Online Video 1). The TNC complexes of AKO mice were smaller and enclosed a significantly reduced number of CD3⁺ thymocytes (FIG. 7D). Moreover, compared to those of AKO mice, the TNC complexes of WT mice showed an increased expression level and a distinct distribution pattern of the thymus-specific proteasome subunit β5t (FIG. 7D). Thymus tissues were collected from WT and AKO mice to examine the microenvironment by immunofluorescence staining of the epithelial markers. While there were no significant changes in the distribution of p63 and galectin-3, the β5t signals showed a clear boundary at the cortico-medullar junction in AKO thymus (Supplementary FIG. 5). Compared to WT thymus, the number of cells co-stained with antibodies against β5t and cytokeratin-5 was significantly decreased (Supplementary FIG. 5).

The results conjointly indicate that adiponectin-expressing tTreg precursors play important roles in the selection and development of T lymphocytes in thymus, particularly within the TNC complexes.

### 7.6 Adiponectin Regulates the Expression and Distribution of CD100

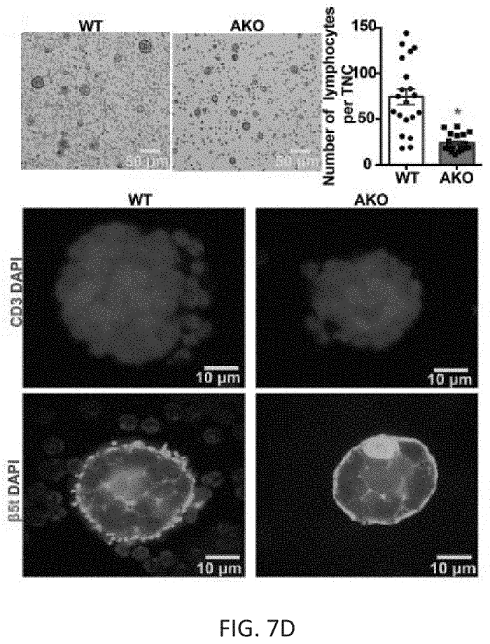

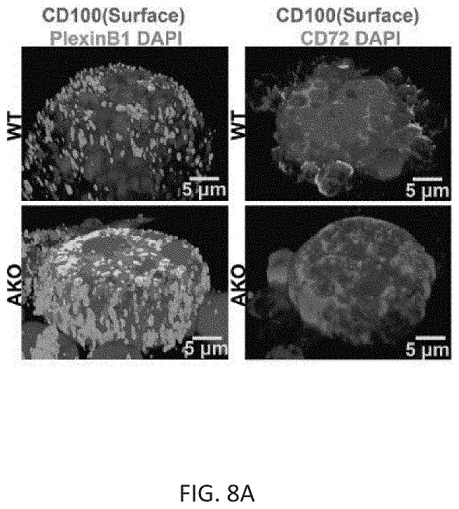

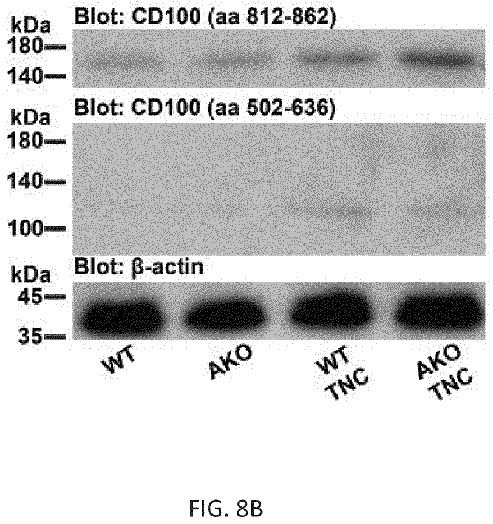

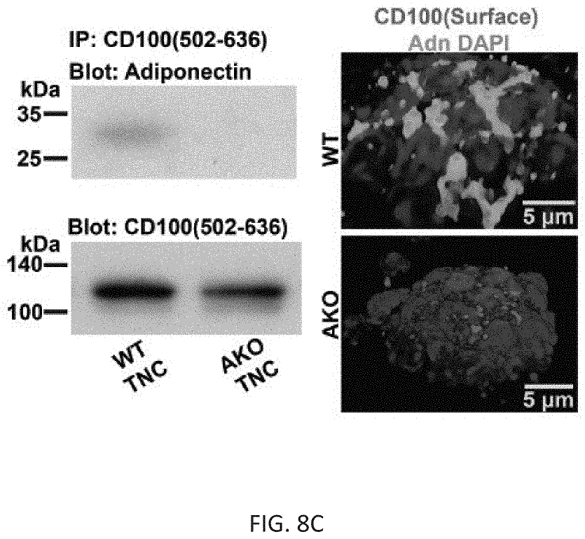

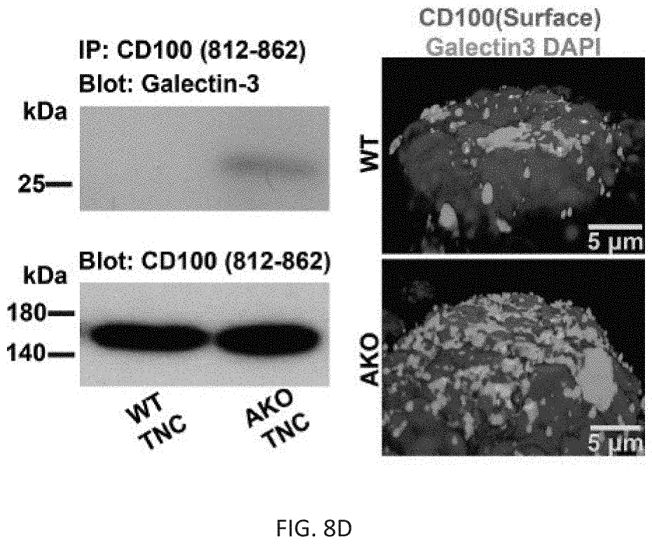

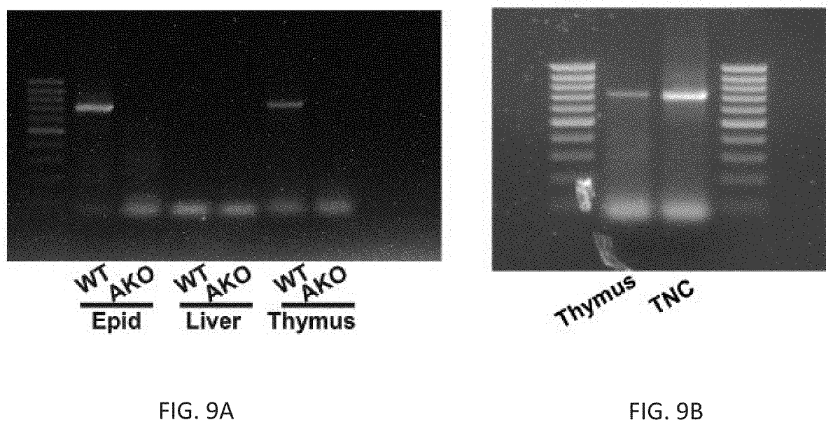

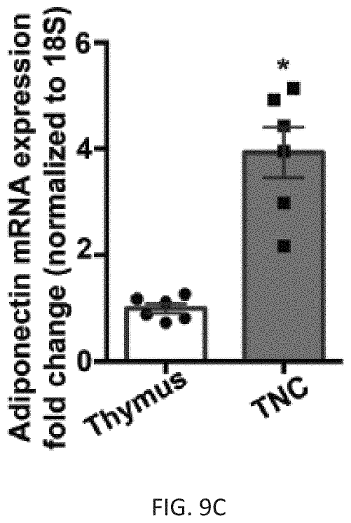

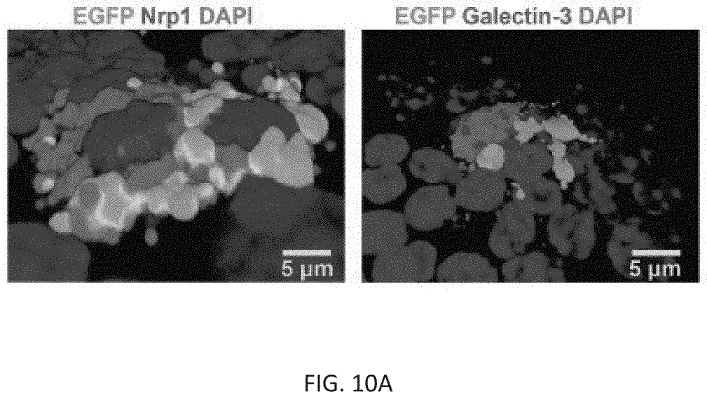

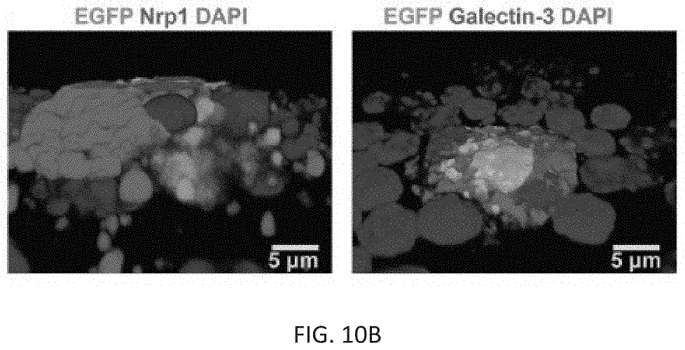

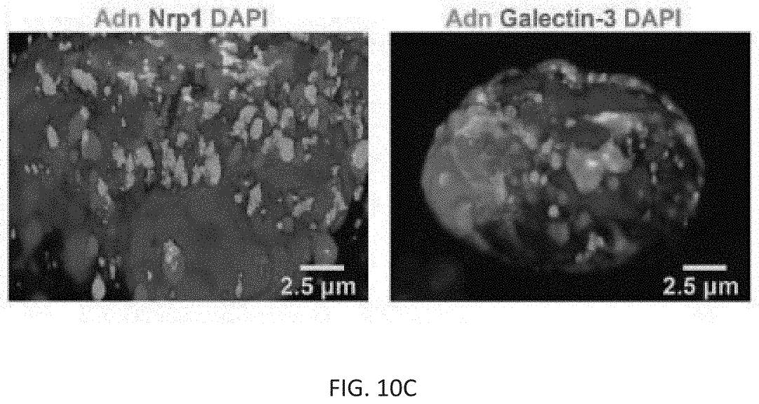

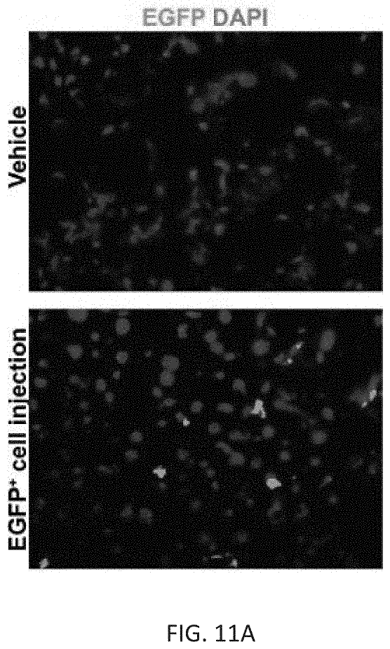

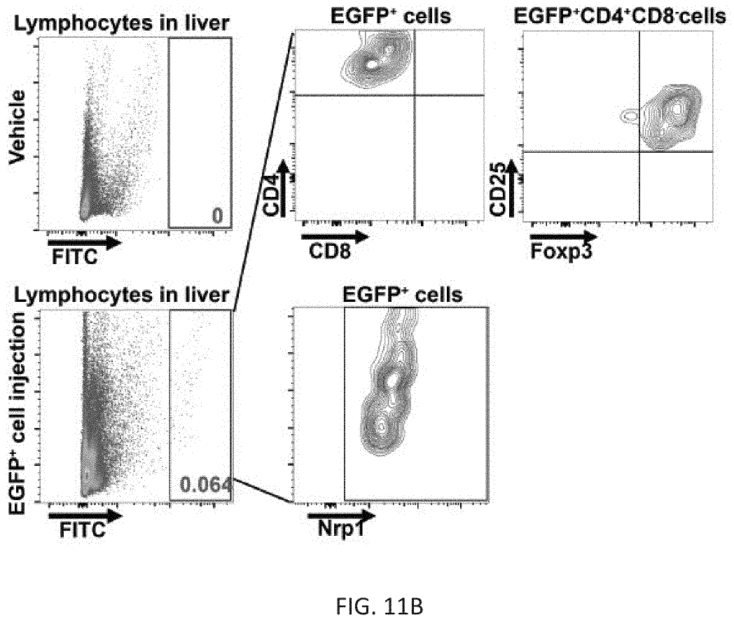

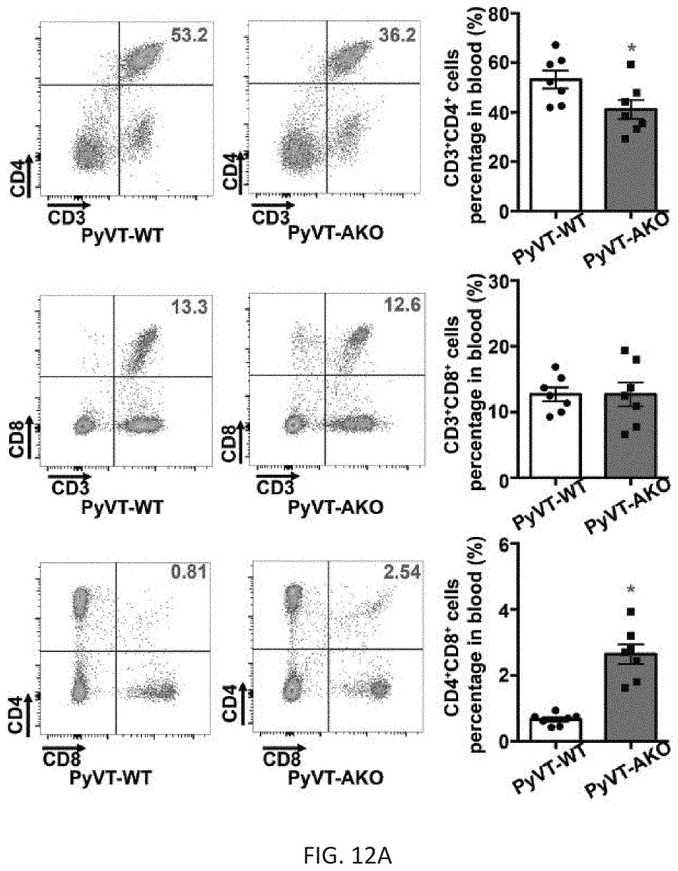

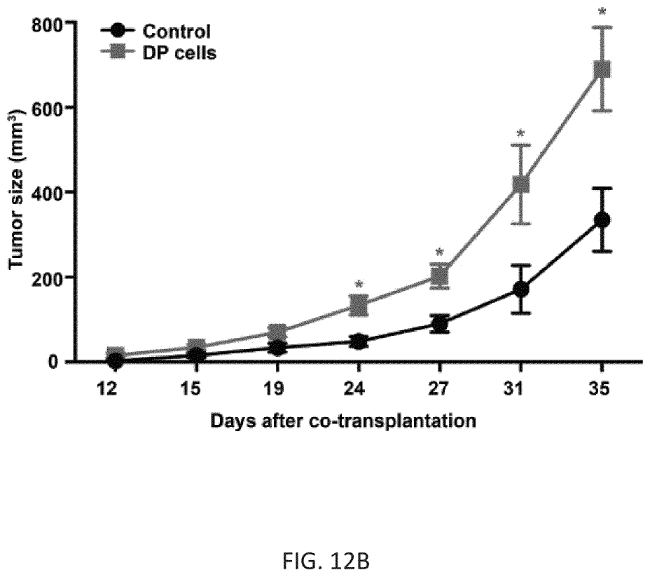

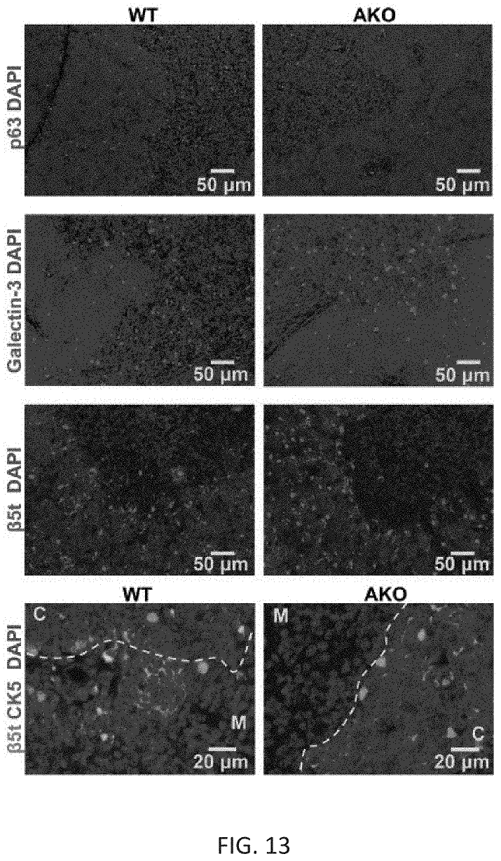

CD100, also known as semaphorin 4D, is a leukocyte cell surface glycoprotein and the first semaphorin member characterized in the immune system ^{(Furuyama et al., 1996)}. The protein was detected in the TNC complexes of WT and AKO mice, however, with significantly different patterns of distribution (FIG. 8A). In TNC complexes isolated from WT thymus, CD100 exhibited distinct close associations with its high affinity receptor plexin B 1^{(Zhang et al., 2013)}. The interactions with CD72, a low affinity receptor of CD100 ^{(Wu and Bondada, 2009)}, were also detectable at the periphery region of TNC complexes (FIG. 8A, top). In TNC complexes isolated from AKO thymus, CD100 was not only distributed around the plexin B1 molecule, but also produced as a diffusible semaphorin filling in the space between the thymocytes and the cage-like structures formed by the epithelial plasma membrane (FIG. 8A, bottom). There were hardly any co-localization signals between CD100 and CD72 within the TNC complexes of AKO mice.

Using the polyclonal antibody recognizing the fragment between amino acid 812 and 862, a 150-kDa CD100 was detected in both thymus and TNC complexes isolated from the WT and AKO mice (FIG. 8B, top). Using the polyclonal antibody recognizing the fragment between amino acid 502 and 636, in addition to the weak 150-kDa band, a 120-kDa CD100 was detected in the TNC complexes isolated from both WT and AKO mice (FIG. 8B, middle). However, the protein amount of the 120-kDa CD100 was significantly decreased in AKO TNC complexes. Immunoprecipitation was performed using the above two polyclonal antibodies recognizing different regions of CD100 (FIGS. 8, C and D). The results demonstrated that adiponectin bound to the extracellular domain of CD100 (FIG. 8C, top), consistent with the immunofluorescent co-staining results in TNC complexes isolated from WT mice (FIG. 8C). By contrast, the protein-protein interactions between CD100 and galectin-3 were only detectable in TNC samples isolated from AKO, but not in the preparations of WT mice (FIG. 8D). The extensive co-localization signals between CD100 and galectin-3 were further confirmed by immunofluorescent staining (FIG. 8D).

### 8. DISCUSSION

Results of the present study demonstrate that adiponectin is expressed in the thymus, by a population of Treg precursors. After tail vein injection, these cells rapidly resided within the TNC complexes and differentiated into tTreg. Adoptive transfer of adiponectin-expressing thymocytes not only effectively prevented HFD-induced obesity, insulin resistance and non-alcoholic fatty liver injuries in WT mice, but also inhibited the breast cancer development in MMTV-PyVT mice.

Due to the very low number of early lymphocyte progenitors, it is difficult to further characterize these non-adipocyte adiponectin-expressing cells. Here, the results showed that adiponectin-expressing thymocytes are of hematopoietic origin, but differentiate and mature into tTreg within the lymphoepithelial TNC. The majority of Treg cells are differentiated and produced in the thymus, from CD25^{hi}CD4⁺CD8⁻ precursors^{(Owen et al., 2019)}. However, the precise signals that divert thymocytes into Treg subsets remain largely undefined. Here, the results demonstrate that the adiponectin-expressing tTreg are likely to differentiate from the CD117⁺CD4⁺CD25⁺ precursors via the CD4⁺CD8⁺ DP stage in the thymus. After adoptive transfer, about 40% of these cells differentiate into mature tTreg in the WT thymus. The development of adiponectin-expressing tTreg is defective in AKO thymus, despite that the precursors exhibited similar capacities to enter the TNC complexes. At 15-days after adoptive transfer, the total number of adiponectin-expressing tTreg is less than 10% of those in WT thymus. However, the percentage composition of EGFP⁺CD4⁺CD8⁺ DP cells is significantly higher in AKO thymus than that of WT thymus, indicating that adiponectin is involved in the regulation of the maturation of tTreg from DP cells within the TNC complexes.

The presence or absence of adiponectin affect the cell-cell interactions of lymphocytes within TNC complexes. The majority of circulating Treg cells are generated in thymus, with only a small amount of peripheral Treg (pTreg) cells induced from CD4⁺CD25⁻ naïve T-cells. Here, the results demonstrate that after adoptive transfer, adiponectin-expressing Treg cells rapidly enter thymus but not liver or adipose tissues of WT mice. Treatment with adiponectin-expressing tTreg precursors protected WT mice from HFD-induced metabolic abnormalities. However, the same treatment in adiponectin-deficient mice (AKO) did not elicit any protective effects (data not shown). Treatment with adiponectin-expressing tTreg cells leads to an increase of Treg and a decrease of Th17 cells in the liver, which at least partly contribute to the prevention of HFD-induced hepatosteatosis and inflammation in WT mice.

The present results demonstrate that without adiponectin, the selection function of TNC complexes is impaired, leading to the escape of immature CD4⁺CD8⁺ thymocytes from thymus and their release into the circulation to facilitate tumor development in PyVT-AKO mice. Treatment with adiponectin-expressing tTreg precursors facilitates the selection and development of DP lymphocytes in TNC complexes, thus preventing the release of immature CD4⁺CD8⁺ thymocytes in the circulation. Apart from the cell-autonomous role in tTreg maturation, adiponectin promotes T-cell selection and facilitates the production of CD3⁺ T-cells in a paracrine manner within the TNC complexes. In tumors of PyVT-WT mice treated with adiponectin-expressing tTreg precursors, two populations of Treg are detected with either CD25^{hi} or CD25^{low} expressions. Taken together, the results demonstrate that adiponectin-expressing tTreg precursors elicit anti-tumorigenic activity at least partly by suppressing the production and/or release of immature CD4⁺CD8⁺ cells from thymus, as well as promoting systemic T-cell homeostasis.

In summary, the results of the present study collectively suggest that adiponectin-expressing tTreg exert the anti-inflammatory, hepatoprotective and anti-tumorigenic activity.

### REFERENCES

Achari, A. E., and Jain, S. K. (2017). Adiponectin, a Therapeutic Target for Obesity, Diabetes, and Endothelial Dysfunction. Int J Mol Sci 18.

Arita, Y., Kihara, S., Ouchi, N., Takahashi, M., Maeda, K., Miyagawa, J., Hotta, K., Shimomura, I., Nakamura, T., Miyaoka, K., et al. (1999). Paradoxical decrease of an adipose-specific protein, adiponectin, in obesity. Biochem Biophys Res Commun 257, 79-83.

Burocchi, A., Colombo, M. P., and Piconese, S. (2013). Convergences and divergences of thymus- and peripherally derived regulatory T cells in cancer. Front Immunol 4, 247.

Burzyn, D., Benoist, C., and Mathis, D. (2013). Regulatory T cells in nonlymphoid tissues. Nat Immunol 14, 1007-1013.

Campbell, D. J., and Koch, M. A. (2011). Phenotypical and functional specialization of FOXP3+ regulatory T cells. Nat Rev Immunol 11, 119-130.

Caramalho, I., Nunes-Cabaco, H., Foxall, R. B., and Sousa, A. E. (2015). Regulatory T-Cell development in the human thymus. Front Immunol 6, 395.

Crawford, L. J., Peake, R., Price, S., Morris, T. C., and Irvine, A. E. (2010). Adiponectin is produced by lymphocytes and is a negative regulator of granulopoiesis. J Leukoc Biol 88, 807-811.

Delgoffe, G. M., Woo, S. R., Turnis, M. E., Gravano, D. M., Guy, C., Overacre, A. E., Bettini, M. L., Vogel, P., Finkelstein, D., Bonnevier, J., et al. (2013). Stability and function of regulatory T cells is maintained by a neuropilin-l-semaphorin-4a axis. Nature 501, 252-256.

DiMascio, L., Voermans, C., Uqoezwa, M., Duncan, A., Lu, D., Wu, J., Sankar, U., and Reya, T. (2007). Identification of adiponectin as a novel hemopoietic stem cell growth factor. J Immunol 178, 3511-3520.

Feuerer, M., Hill, J. A., Mathis, D., and Benoist, C. (2009). Foxp3+ regulatory T cells: differentiation, specification, subphenotypes. Nat Immunol 10, 689-695.

Funahashi, T., Nakamura, T., Shimomura, I., Maeda, K., Kuriyama, H., Takahashi, M., Arita, Y., Kihara, S., and Matsuzawa, Y. (1999). Role of adipocytokines on the pathogenesis of atherosclerosis in visceral obesity. Intern Med 38, 202-206.

Furuyama, T., Inagaki, S., Kosugi, A., Noda, S., Saitoh, S., Ogata, M., Iwahashi, Y., Miyazaki, N., Hamaoka, T., and Tohyama, M. (1996). Identification of a novel transmembrane semaphorin expressed on lymphocytes. The Journal of biological chemistry 271, 33376-33381.

Gratz, I. K., and Campbell, D. J. (2014). Organ-specific and memory treg cells: specificity, development, function, and maintenance. Front Immunol 5, 333.

Guyden, J. C., Martinez, M., Chilukuri, R. V., Reid, V., Kelly, F., and Samms, M. O. (2015). Thymic Nurse Cells Participate in Heterotypic Internalization and Repertoire Selection of Immature Thymocytes; Their Removal from the Thymus of Autoimmune Animals May be Important to Disease Etiology. Curr Mol Med 15, 828-835.

Hampe, L., Xu, C., Harris, P. W., Chen, J., Liu, M., Middleditch, M., Radjainia, M., Wang, Y., and Mitra, A. K. (2017). Synthetic peptides designed to modulate adiponectin assembly improve obesity-related metabolic disorders. British journal of pharmacology 174, 4478-4492.

Hendrix, T. M., Chilukuri, R. V., Martinez, M., Olushoga, Z., Blake, A., Brohi, M., Walker, C., Samms, M., and Guyden, J. C. (2010). Thymic nurse cells exhibit epithelial progenitor phenotype and create unique extra-cytoplasmic membrane space for thymocyte selection. Cell Immunol 261, 81-92.

Herold, C., Elhabazi, A., Bismuth, G., Bensussan, A., and Boumsell, L. (1996). CD100 is associated with CD45 at the surface of human T lymphocytes. Role in T cell homotypic adhesion. J Immunol 157, 5262-5268.

Hui, X., Gu, P., Zhang, J., Nie, T., Pan, Y., Wu, D., Feng, T., Zhong, C., Wang, Y., Lam, K. S., and Xu, A. (2015). Adiponectin Enhances Cold-Induced Browning of Subcutaneous Adipose Tissue via Promoting M2 Macrophage Proliferation. Cell Metab 22, 279-290.

Jasinski-Bergner, S., Buttner, M., Quandt, D., Seliger, B., and Kielstein, H. (2017). Adiponectin and its receptors are differentially expressed in human tissues and cell lines of distinct origin. Obes Facts 10, 569-583.

Jung, M. K., Kwak, J. E., and Shin, E. C. (2017). IL-17A-Producing Foxp3(+) Regulatory T Cells and Human Diseases. Immune Netw 17, 276-286.

Klein, L., Robey, E. A., and Hsieh, C. S. (2019). Central CD4(+) T cell tolerance: deletion versus regulatory T cell differentiation. Nat Rev Immunol 19, 7-18.

Koizumi, S. I., and Ishikawa, H. (2019). Transcriptional Regulation of Differentiation and Functions of Effector T Regulatory Cells. Cells 8.

Krueger, A., Zietara, N., and Lyszkiewicz, M. (2017). T Cell Development by the Numbers. Trends in immunology 38, 128-139.

Kumanogoh, A., Shikina, T., Watanabe, C., Takegahara, N., Suzuki, K., Yamamoto, M., Takamatsu, H., Prasad, D. V., Mizui, M., Toyofuku, T., et al. (2005). Requirement for CD100-CD72 interactions in fine-tuning of B-cell antigen receptor signaling and homeostatic maintenance of the B-cell compartment. Int Immunol 17, 1277-1282.

Lam, J. B., Chow, K. H., Xu, A., Lam, K. S., Liu, J., Wong, N. S., Moon, R. T., Shepherd, P. R., Cooper, G. J., and Wang, Y. (2009). Adiponectin haploinsufficiency promotes mammary tumor development in MMTV-PyVT mice by modulation of phosphatase and tensin homolog activities. PLoS One 4, e4968.

Lee, H. M., and Hsieh, C. S. (2009). Rare development of Foxp3+ thymocytes in the CD4+CD8+ subset. J Immunol 183, 2261-2266.

Li, D., Cao, H., Chen, J., Liu, M., and Wang, Y. (2017). Adiponectin-based therapeutics in cancer treatment. Frontiers in Clinical Drug Research-Anti-Cancer Agents 4, 272-313.

Lin, Z., Tian, H., Lam, K. S., Lin, S., Hoo, R. C., Konishi, M., Itoh, N., Wang, Y., Bornstein, S. R., and Xu, A. (2013). Adiponectin mediates the metabolic effects of FGF21 on glucose homeostasis and insulin sensitivity in mice. Cell metabolism 17, 779-789.

Liu, J., Xu, A., Lam, K. S., Wong, N. S., Chen, J., Shepherd, P. R., and Wang, Y. (2013). Cholesterol-induced mammary tumorigenesis is enhanced by adiponectin deficiency: role of LDL receptor upregulation. Oncotarget 4, 1804-1818.

Luo, Y., and Liu, M. (2016). Adiponectin: a versatile player of innate immunity. J Mol Cell Biol 8, 120-128.

Malhotra, D., Linehan, J. L., Dileepan, T., Lee, Y. J., Purtha, W. E., Lu, J. V., Nelson, R. W., Fife, B. T., Orr, H. T., Anderson, M. S., et al. (2016). Tolerance is established in polyclonal CD4(+) T cells by distinct mechanisms, according to self-peptide expression patterns. Nat Immunol 17, 187-195.

Nakagawa, H., Sido, J. M., Reyes, E. E., Kiers, V., Cantor, H., and Kim, H. J. (2016). Instability of Helios-deficient Tregs is associated with conversion to a T-effector phenotype and enhanced antitumor immunity. Proc Natl Acad Sci U S A 113, 6248-6253.

Nakagawa, Y., Ohigashi, I., Nitta, T., Sakata, M., Tanaka, K., Murata, S., Kanagawa, O., and Takahama, Y. (2012). Thymic nurse cells provide microenvironment for secondary T cell receptor alpha rearrangement in cortical thymocytes. Proc Natl Acad Sci USA 109, 20572-20577.

Okeke, E. B., and Uzonna, J. E. (2019). The Pivotal Role of Regulatory T Cells in the Regulation of Innate Immune Cells. Front Immunol 10, 680.

Overacre-Delgoffe, A. E., Chikina, M., Dadey, R. E., Yano, H., Brunazzi, E. A., Shayan, G., Horne, W., Moskovitz, J. M., Kolls, J. K., Sander, C., et al. (2017). Interferon-gamma Drives Treg Fragility to Promote Anti-tumor Immunity. Cell 169, 1130-1141 e1111.

Owen, D. L., Mahmud, S. A., Sjaastad, L. E., Williams, J. B., Spanier, J. A., Simeonov, D. R., Ruscher, R., Huang, W., Proekt, I., Miller, C. N., et al. (2019). Thymic regulatory T cells arise via two distinct developmental programs. Nat Immunol 20, 195-205.

Perry, J. S., and Hsieh, C. S. (2016). Development of T-cell tolerance utilizes both cell-autonomous and cooperative presentation of self-antigen. Immunol Rev 271, 141-155.

Porritt, H. E., Rumfelt, L. L., Tabrizifard, S., Schmitt, T. M., Zuniga-Pflucker, J. C., and Petrie, H. T. (2004). Heterogeneity among DN1 prothymocytes reveals multiple progenitors with different capacities to generate T cell and non-T cell lineages. Immunity 20, 735-745.

Rodriguez-Perea, A. L., Arcia, E. D., Rueda, C. M., and Velilla, P. A. (2016). Phenotypical characterization of regulatory T cells in humans and rodents. Clin Exp Immunol 185, 281-291.

Saito, T., Nishikawa, H., Wada, H., Nagano, Y., Sugiyama, D., Atarashi, K., Maeda, Y., Hamaguchi, M., Ohkura, N., Sato, E., et al. (2016). Two FOXP3(+)CD4(+) T cell subpopulations distinctly control the prognosis of colorectal cancers. Nat Med 22, 679-684.

Smigiel, K. S., Srivastava, S., Stolley, J. M., and Campbell, D. J. (2014). Regulatory T-cell homeostasis: steady-state maintenance and modulation during inflammation. Immunol Rev 259, 40-59.

Stern, J. H., Rutkowski, J. M., and Scherer, P. E. (2016). Adiponectin, leptin, and fatty acids in the maintenance of metabolic homeostasis through adipose tissue crosstalk. Cell Metab 23, 770-784.

Takahama, Y., Ohigashi, I., Baik, S., and Anderson, G. (2017). Generation of diversity in thymic epithelial cells. Nat Rev Immunol 17, 295-305.

Van Herck, M. A., Weyler, J., Kwanten, W. J., Dirinck, E. L., De Winter, B. Y., Francque, S. M., and Vonghia, L. (2019). The Differential Roles of T Cells in Non-alcoholic Fatty Liver Disease and Obesity. Front Immunol 10, 82.

Wang, Y., Lam, J. B., Lam, K. S., Liu, J., Lam, M. C., Hoo, R. L., Wu, D., Cooper, G. J., and Xu, A. (2006a). Adiponectin modulates the glycogen synthase kinase-3beta/beta-catenin signaling pathway and attenuates mammary tumorigenesis of MDA-MB-231 cells in nude mice. Cancer Res 66, 11462-11470.

Wang, Y., Lam, K. S., Chan, L., Chan, K. W., Lam, J. B., Lam, M. C., Hoo, R. C., Mak, W. W., Cooper, G. J., and Xu, A. (2006b). Post-translational modifications of the four conserved lysine residues within the collagenous domain of adiponectin are required for the formation of its high molecular weight oligomeric complex. J Biol Chem 281, 16391-16400.

Wang, Y., Lam, K. S., Xu, J. Y., Lu, G., Xu, L. Y., Cooper, G. J., and Xu, A. (2005). Adiponectin inhibits cell proliferation by interacting with several growth factors in an oligomerization-dependent manner. J Biol Chem 280, 18341-18347.

Wang, Y., Lam, K. S., Yau, M. H., and Xu, A. (2008). Post-translational modifications of adiponectin: mechanisms and functional implications. Biochem J 409, 623-633.

Wang, Y., Xu, A., Knight, C., Xu, L. Y., and Cooper, G. J. (2002). Hydroxylation and glycosylation of the four conserved lysine residues in the collagenous domain of adiponectin. Potential role in the modulation of its insulin-sensitizing activity. J Biol Chem 277, 19521-19529.

Wang, Y., Zhou, M., Lam, K. S., and Xu, A. (2009). Protective roles of adiponectin in obesity-related fatty liver diseases: mechanisms and therapeutic implications. Arq Bras Endocrinol Metabol 53, 201-212.

Wang, Z. V., and Scherer, P. E. (2016). Adiponectin, the past two decades. J Mol Cell Biol 8, 93-100.

Weissler, K. A., and Caton, A. J. (2014). The role of T-cell receptor recognition of peptide:MHC complexes in the formation and activity of Foxp3(+) regulatory T cells. Immunol Rev 259,11-22.

Wekerle, H., Ketelsen, U., and Ernst, M. (1980). Thymic nurse cells. Lymphoepithelial cell complexes in murine thymuses: morphological and serological characterization. Journal of Experimental Medicine 151, 925-944.

Winer, S., Chan, Y., Paltser, G., Truong, D., Tsui, H., Bahrami, J., Dorfman, R., Wang, Y., Zielenski, J., Mastronardi, F., et al. (2009). Normalization of obesity-associated insulin resistance through immunotherapy. Nat Med 15, 921-929.

Wu, H. J., and Bondada, S. (2009). CD72, a coreceptor with both positive and negative effects on B lymphocyte development and function. J Clin Immunol 29, 12-21.

Xing, Y., and Hogquist, K. A. (2014). Isolation, identification, and purification of murine thymic epithelial cells. Journal of visualized experiments: JoVE.

Xu, A., Wang, Y., Keshaw, H., Xu, L. Y., Lam, K. S., and Cooper, G. J. (2003). The fat-derived hormone adiponectin alleviates alcoholic and nonalcoholic fatty liver diseases in mice. J Clin Invest 112, 91-100.

Xu, C., Cai, Y., Fan, P., Bai, B., Chen, J., Deng, H. B., Che, C. M., Xu, A., Vanhoutte, P. M., and Wang, Y. (2015). Calorie Restriction Prevents Metabolic Aging Caused by Abnormal SIRT1 Function in Adipose Tissues. Diabetes 64, 1576-1590.

Yamano, T., Nedjic, J., Hinterberger, M., Steinert, M., Koser, S., Pinto, S., Gerdes, N., Lutgens, E., Ishimaru, N., Busslinger, M., et al. (2015). Thymic B cells are licensed to present self antigens for central T cell tolerance induction. Immunity 42, 1048-1061.

Yamauchi, T., Kamon, J., Waki, H., Terauchi, Y., Kubota, N., Hara, K., Mori, Y., Ide, T., Murakami, K., Tsuboyama-Kasaoka, N., et al. (2001). The fat-derived hormone adiponectin reverses insulin resistance associated with both lipoatrophy and obesity. Nat Med 7, 941-946.

Yokota, T., Meka, C. S., Kouro, T., Medina, K. L., Igarashi, H., Takahashi, M., Oritani, K., Funahashi, T., Tomiyama, Y., Matsuzawa, Y., and Kincade, P. W. (2003). Adiponectin, a fat cell product, influences the earliest lymphocyte precursors in bone marrow cultures by activation of the cyclooxygenase-prostaglandin pathway in stromal cells. J Immunol 171, 5091-5099.

Yokota, T., Oritani, K., Takahashi, I., Ishikawa, J., Matsuyama, A., Ouchi, N., Kihara, S., Funahashi, T., Tenner, A. J., Tomiyama, Y., and Matsuzawa, Y. (2000). Adiponectin, a new member of the family of soluble defense collagens, negatively regulates the growth of myelomonocytic progenitors and the functions of macrophages. Blood 96, 1723-1732.

Zhang, Y., Liu, B., Ma, Y., and Jin, B. (2013). Sema 4D/CD100-plexin B is a multifunctional counter-receptor. Cell Mol Immunol 10, 97-98.

Zhou, M., Xu, A., Lam, K. S., Tam, P. K., Che, C.-M., Chan, L., Lee, I.-K., Wu, D., and Wang, Y. (2010). Rosiglitazone promotes fatty acyl CoA accumulation and excessive glycogen storage in livers of mice without adiponectin. Journal of hepatology 53, 1108-1116.

Zhou, M., Xu, A., Tam, P. K., Lam, K. S., Chan, L., Hoo, R. L., Liu, J., Chow, K. H., and Wang, Y. (2008). Mitochondrial dysfunction contributes to the increased vulnerabilities of adiponectin knockout mice to liver injury. Hepatology 48, 1087-1096.

Fujimaki W. et al. (2008). Comparative Study of Regulatory T cell Function of Human CD25 CD4 T cells from Thymocytes, cord blood and adult peripheral blood. Clin. Dev. Immunol 2008, article ID 305859.

MacDonald K. N. et al. (2019) Methods to manufacture regulatory T cells for cell therapy. Clin. & Exp. Immunol J. Trans Immunol. 197: 52-63.

Rosenberg, S. A. et al. (2015) Adoptive cell transfer as personalized immunotherapy for human cancer. Science (348) 6230: 62-68.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

1. A method of isolating adiponectin-expressing thymocytes from a subject comprising the steps of: (i) obtaining a portion of thymus from the subject; (ii) enriching thymic nurse cell (TNC) complexes from the thymus; (iii) preparing single-cell suspension from the TNC; and (iv) sorting adiponectin-expressing thymocytes from the single-cell suspension.

2. The method of claim 1 wherein the adiponectin-expressing thymocytes are characterized by high expression of CD117, CD4⁺, CD25⁺, and low expression of CD8⁻.

3. A method of treating or preventing an autoimmune, metabolic, hyperproliferative or inflammatory disease in a subject, said method comprising the steps of: (i) obtaining a portion of a thymus from a subject; (ii) enriching thymic nurse cell (TNC) complexes from the thymus; (iii) preparing single-cell suspension from the TNC; (iv) sorting adiponectin-expressing thymocytes from the single-cell suspension; and (v) administering an effective amount of adiponectin-expressing thymocytes to a subject in need thereof.

4. The method of claim 3 wherein the thymus of step (i) was obtained from the same subject to be treated.

5. The method of claim 3 wherein the thymus of step (i) was obtained from a different subject to be treated.

6. The method of claim 3 wherein the method further comprising a step of culturing the adiponectin-expressing thymocytes after step (iv).

7. The method of claim 3 wherein after the treatment, the subject has one or more of the following outcomes: (i) maintaining immune homeostatis; (ii) facilitating self-tolerance; (iii) inhibiting mammary tumor; (iv) inhibiting dietary obesity-induced gain of body fat mass; (v) improved glucose tolerance; (vi) reducing circulating lipid levels; (vii) enhanced energy expenditure; (viii) reducing atherosclerosis; (ix) hepatoprotection; (x) improved insulin sensitivity; (xi) enhanced oxygen consumption; (xii) enhanced CO₂ production; (xiii) reduced circulating triglyceride and cholesterol levels; (xiv) reduced plasma concentrations of alanine transaminase (ALT); and (xv) reduced level of aspartate transaminase (AST).

8. The method of claim 3 wherein the effective amount of adiponectin-expressing thymocytes is about 30,000 to about 500,000 cells.

9. A thymic nurse cell (TNC) complex comprising isolated adiponectin-expressing thymocytes from an Adn-Cre/ROSA^{mT/mG} mouse wherein said adiponectin-expressing thymocytes express EGFP.

10. The isolated thymocytes of claim 9 wherein the thymocytes are characterized by high expression of CD117, CD4⁺, CD25⁺, and low expression of CD8⁻.

11. A method of producing adiponectin-rich thymocytes from an Adn-Cre/ROSA^{mT/mG} mouse comprising the steps of: (i) obtaining a portion of a thymus from the mouse; (ii) enriching thymic nurse cell (TNC) complexes from the thymus; (iii) preparing single-cell suspension from the TNC; (iv) sorting adiponectin-expressing thymocytes EGFP cells from the single-cell suspension.

12. A transgenic mouse Adn-Cre/ROSA^{mT/mG}.

13. A method of treating or preventing an autoimmune, metabolic, hyperproliferative or inflammatory disease in a subject, said method comprising the steps of administering a therapeutically effective amount of thymocytes comprising a population of adiponectin-expressing regulatory T cell (tReg) to a subject in need thereof.

14. The method according to claim 13, wherein the adiponectin-expressing thymocytes are obtained by (i) obtaining a portion of a thymus from a subject; (ii) enriching thymic nurse cell (TNC) complexes from the thymus; (iii) preparing single-cell suspension from the TNC; and (iv) sorting adiponectin-expressing thymocytes from the single-cell suspension.

15. The method of claim 13, wherein adiponectin-expressing tReg precursors differentiate into regulatory T cells in the subject.

16. The method according to claim 13, wherein the metabolic disease is selected from the group consisting of metabolic syndrome, type-I diabetes mellitus, type-2 diabetes, obesity, diseases associated with an abnormal fat metabolism, gout disease (metabolic arthritis), hyperglycemia, hyperinsulinemia, insulin resistance, elevated blood levels of fatty acids or glycerol, syndrome X, and diabetic complications.

17. The method according to claim 13, wherein the autoimmune or inflammatory disease is selected from the group consisting of wherein the autoimmune or inflammatory disease is selected from the group consisting of rheumatoid arthritis, multiple sclerosis, autoimmune hemolytic anemia, autoimmune oophoritis, autoimmune thyroiditis, autoimmune uveoretinitis, Crohn's disease, chronic immune thrombocytopenic purpura, colitis, contact sensitivity disease, type-1 diabetes mellitus, Graves disease, Guillain-Barre's syndrome, Hashimoto's disease, idiopathic myxedema, myasthenia gravis, psoriasis, pemphigus vulgaris, systemic lupus erythematosus, scleroderma, Reynaud's syndrome, Sjorgen's syndrome, autoimmune myocarditis, inflammatory bowel disease, Amyotrophic Lateral Sclerosis (ALS), Neuromyelitis Optica, Idiopathic Thrombocytopenic Purpura, Thrombotic Thrombocytopenic Purpura, Membranous Nephropathy, Bullous Phemphigoid, Phemphigus Vulgaris, Celiac disease, and ulcerative colitis.

18. The method according to claim 13, wherein the hyperproliferative disease is cancer, including metastases thereof.

19. The method according to claim 18, wherein the cancer is selected from the group consisting of ovarian cancer, prostate cancer, breast cancer, skin cancer, melanoma, colon cancer, lung cancer, pancreatic cancer, gastric cancer, bladder cancer, Ewing's sarcoma, lymphoma, leukemia, multiple myeloma, head and neck cancer, kidney cancer, bone cancer, liver cancer and thyroid cancer, including metastases thereof.

20. The method of claim 13 wherein the thymocytes are obtained from the same subject to be treated.

21. The method of claim 13 wherein the thymocytes are obtained from a different subject to be treated.

22. The method of claim 13 wherein after the treatment, the subject has one or more of the following outcomes: (i) maintaining immune homeostatis; (ii) facilitating self-tolerance; (iii) inhibiting mammary tumor; (iv) inhibiting dietary obesity-induced gain of body fat mass; (v) improved glucose tolerance; (vi) reducing circulating lipid levels; (vii) enhanced energy expenditure; (viii) reducing atherosclerosis; (ix) hepatoprotection; (x) improved insulin sensitivity; (xi) enhanced oxygen consumption; (xii) enhanced CO2 production; (xiii) reduced circulating triglyceride and cholesterol levels; (xiv) reduced plasma concentrations of alanine transaminase (ALT); and (xv) reduced level of aspartate transaminase (AST).

23. The method of claim 13 wherein the effective amount of adiponectin-expressing thymocytes is about 30,000 to about 500,000 cells.

24. The method of claim 14 wherein the method further comprising a step of culturing the adiponectin-expressing thymocytes after step (iv).


SEQUENCE LISTING (31 sequences)

SEQ ID NO: 1 - DNA, 21 bp, Artificial Sequence
  Adipoq
ccagagaacaacgaacaagga

SEQ ID NO: 2 - DNA, 20 bp, Artificial Sequence
  Adipoq
cgaatgggtacattgggaac

SEQ ID NO: 3 - DNA, 20 bp, Artificial Sequence
  Neo
tgaatgaactgcaggacgag

SEQ ID NO: 4 - DNA, 20 bp, Artificial Sequence
  Neo
atactttctcggcaggagca

SEQ ID NO: 5 - DNA, 22 bp, Artificial Sequence
  MMTV-PvVT
ggaagcaagtacttcacaaggg

SEQ ID NO: 6 - DNA, 21 bp, Artificial Sequence
  MMTV-PyVT
ggaaagtcactaggagcaggg

SEQ ID NO: 7 - DNA, 20 bp, Artificial Sequence
  Adipoq-cre
ggatgtgccatgtgagtctg

SEQ ID NO: 8 - DNA, 20 bp, Artificial Sequence
  Adipoq-cre
acggacagaagcattttcca

SEQ ID NO: 9 - DNA, 20 bp, Artificial Sequence
  Gt(Rosa)26sor
ctttaagcctgcccagaaga

SEQ ID NO: 10 - DNA, 20 bp, Artificial Sequence
  Neo
tagagcttgcggaacccttc

SEQ ID NO: 11 - DNA, 20 bp, Artificial Sequence
  Wild Type
agggagctgcagtggagtag

SEQ ID NO: 12 - DNA, 23 bp, Artificial Sequence
  Adipoq
aagctctcctgttcctcttaatc

SEQ ID NO: 13 - DNA, 23 bp, Artificial Sequence
  Adipoq
catggtagagaagaaagccagta

SEQ ID NO: 14 - DNA, 21 bp, Artificial Sequence
  Adipoq
tgccgaagatgacgttactac

SEQ ID NO: 15 - DNA, 21 bp, Artificial Sequence
  Adipoq
gtagagtcgttgacgttatct

SEQ ID NO: 16 - DNA, 20 bp, Artificial Sequence
  Adipoq
ctccacccaagggaacttgt

SEQ ID NO: 17 - DNA, 22 bp, Artificial Sequence
  Adipoq
taggaccaagaagacctgcatc

SEQ ID NO: 18 - DNA, 20 bp, Artificial Sequence
  FGF21
tatggatcgcctcactttga

SEQ ID NO: 19 - DNA, 20 bp, Artificial Sequence
  FGF21
ggagtccttctgaggcagac

SEQ ID NO: 20 - DNA, 23 bp, Artificial Sequence
  PPARalpha
gagtgcagcctcagccaagttga

SEQ ID NO: 21 - DNA, 21 bp, Artificial Sequence
  PPARalpha
aggcaggccacagagcgctaa

SEQ ID NO: 22 - DNA, 20 bp, Artificial Sequence
  TNFalpha
tcgtagcaaaccaccaagtg

SEQ ID NO: 23 - DNA, 20 bp, Artificial Sequence
  TNFalpha
agatagcaaatcggctgacg

SEQ ID NO: 24 - DNA, 23 bp, Artificial Sequence
  MCP-1
tgatcccaatgagtaggctggag

SEQ ID NO: 25 - DNA, 23 bp, Artificial Sequence
  MCP-1
atgtctggacccattccttcttg

SEQ ID NO: 26 - DNA, 20 bp, Artificial Sequence
  TGFbeta
tggagcaacatgtggaactc

SEQ ID NO: 27 - DNA, 20 bp, Artificial Sequence
  TGFbeta
cgtcaaaagacagccactca

SEQ ID NO: 28 - DNA, 25 bp, Artificial Sequence
  IL-6
tggagtcacagaaggagtggctaag

SEQ ID NO: 29 - DNA, 25 bp, Artificial Sequence
  IL-6
tctgaccacagtgaggaatgtccac

SEQ ID NO: 30 - DNA, 20 bp, Artificial Sequence
  VEGFalpha
aacgatgaagccctggagtg

SEQ ID NO: 31 - DNA, 19 bp, Artificial Sequence
  VEGFalpha
tgagaggtctggttcccga